(19) United States
(12) Patent Application Publication
(10) Pub. No.: US 2024/0041742 A1
(43) Pub. Date: Feb. 8, 2024

(54) USE OF SYNTHETIC COPOLYPEPTIDE HYDROGELS AS DERMAL FILLERS

(71) Applicant: The Regents of the University of California, Oakland, CA (US)

(72) Inventors: Timothy J. Deming, Los Angeles, CA (US); Michael Delong, Los Angeles, CA (US)

(21) Appl. No.: 18/265,368

(22) PCT Filed: Dec. 3, 2021
(86) PCT No.: PCT/US21/61843

§ 371 Date: Jun. 5, 2023

Related U.S. Application Data
(60) Provisional application No. 63/120,850, filed on Dec. 3, 2020.

(51) Int. Cl. A61K 8/64; A61K 8/04; A61Q 19/08

(52) U.S. Cl. CPC A61K 8/64; A61K 8/042; A61Q 19/08; A61K 2800/91

(57) ABSTRACT

Provided herein are synthetic copolypeptide hydrogel compositions for use as dermal fillers, and methods of treating dermatological conditions using the same.

### RELATED APPLICATION

This application claims the benefit of U.S. Provisional Patent Application No. 63/120,850, filed on Dec. 3, 2020. The contents of this application are hereby incorporated by reference in their entirety.

### GOVERNMENT SUPPORT

This invention was made with government support under Grant Number 1807362, awarded by the National Science Foundation. The government has certain rights in the invention.

### BACKGROUND

The present disclosure generally relates to dermal filler compositions, for example, but not limited to, dermal filler compositions that are effective for treatment of fine lines in skin. Skin aging is a progressive phenomenon, occurs over time and can be affected by lifestyle factors, such as alcohol consumption, tobacco and sun exposure. Aging of the facial skin can be characterized by atrophy, slackening, and fattening. These changes are typically associated with dryness, loss of elasticity, and rough texture. Traditional dermal fillers suffer from a number of drawbacks, such as vascular occlusion or compression, skin necrosis, or blindness. Accordingly, there is a need for better dermal fillers for treating and improving the appearance of aging skin.

### SUMMARY

The present disclosure provides a method of treating fine lines or superficial wrinkles in the skin of a subject, comprising administering a composition into a dermal region of the subject which displays the fine lines or superficial wrinkles, thereby treating the fine lines or superficial wrinkles, wherein the composition comprises a polypeptide hydrogel.

The present disclosure also provides a method of treating a skin condition, comprising administering to an individual suffering from the skin condition a composition, wherein the administration of the composition improves the skin condition, thereby treating the skin condition, wherein the composition comprises a polypeptide hydrogel.

The present disclosure further provides a method of preventing skin wrinkles in a subject, comprising administering to the subject a composition, thereby preventing skin wrinkles, wherein the composition comprises a polypeptide hydrogel.

### BRIEF DESCRIPTION OF THE DRAWINGS

FIG. 1A is an inner addition approach to load the PIC hydrogels into syringes for subsequent injection.

FIG. 1B is a simultaneous addition approach to load the PIC hydrogels into syringes for subsequent injection.

FIG. 1C is a successive addition approach to load the PIC hydrogels into syringes for subsequent injection.

FIG. 1D is a manual loading approach to load the PIC hydrogels into syringes for subsequent injection.

FIG. 2 is an example photograph from an animal of Animal Study #1 in group 2 at day 30 with no observable erythema or irritation at the site of injection (marked with the dark circle).

FIG. 3 is an example photo of a palpable lump on the dorsum of an animal of Animal Study #2 in Group A (Hyaluronic Acid Control) on day 7.

FIG. 4 shows day 0 transillumination from left ear of rabbit 1 (Hyaluronic acid control) demonstrates impeded blood flow and embolus in the central auricular artery (Animal Study #3).

FIG. 5 shows day 0 transillumination from right ear of rabbit 1 (M^{O}A₁₅₅(E/K)₆₅ at 7 wt % in 0.9% NaCl) demonstrates intact blood flow in the central auricular artery without apparent emboli. These results were representative of the day 0 transillumination experiments. All of the hyaluronic acid ears showed emboli with impeded blood flow, while all of the claimed hydrogel filler injected ears remained patent (Animal Study #3).

FIG. 6 is a photo from day 7 from animal 1 depicting ischemic changes in the left ear (hyaluronic acid) compared to right ear (M^{O}A₁₅₅(E/K)₆₅ at 7 wt % in 0.9% NaCl). The ischemic changes are clearly seen as the dusky coloration in the auricular tissue (Animal Study #3).

FIG. 7 is a photo from day 7 of rabbit 3 demonstrates ischemic changes in the right ear (hyaluronic acid), but no ischemic changes in the left ear (M^{O}A₁₈₀(E/K)₇₅ at 7 wt % in 0.9% NaCl). As with the transillumination studies, these changes were consistent and reproducible in the animals. All of the hyaluronic acid ears demonstrated ischemic changes at day 7, while none of the claimed hydrogel filler injected ears demonstrated ischemic changes (Animal Study #3).

FIG. 8 depicts a schematic representation of the assembly process for preparation of polyion complex (M^{O}A)₁₅₅E/Kₓ diblock copolypeptide hydrogels, which are employed in the animals studies recited in Example 2.

FIG. 9 depicts the properties of polysarcosine copolymers used for polyion complex formation.

FIG. 10 depicts the viscosity of several diblock copolypeptide hydrogels.

FIG. 11A depicts a plot of G′ & G″ vs. oscillation stress.

FIG. 11B depicts a plot of G′ & G″ and viscosity vs. frequency.

### DETAILED DESCRIPTION

The disclosure relates to compositions comprising synthetic polypeptide hydrogel useful for treating fine lines or superficial wrinkles in the skin, for treating or preventing skin wrinkles, or for treating other skin conditions. In certain embodiments, the compositions comprise synthetic diblock copolypeptides having oppositely charged ionic segments, which form β-sheet structured hydrogel assemblies via polyion complexation when mixed in aqueous media.

### Methods

In one embodiment, the disclosure relates to a method of treating fine lines or superficial wrinkles in the skin of a subject, comprising administering a composition described herein into a dermal region of the subject.

In some embodiments, the dermal region is a tear trough region, a glabellar line, a periorbital region, or a forehead region.

In a second embodiment, the disclosure relates to a method of treating a skin condition, comprising administering to an individual suffering from a skin condition a composition described herein, wherein the administration of the composition improves the skin condition, thereby treating the skin condition.

In some embodiments, the skin condition is skin dehydration, skin roughness, a lack of skin tautness, a skin stretch line or mark, or skin wrinkles.

In a third embodiment, the disclosure relates to a method of treating skin dehydration, comprising administering to an individual suffering from skin dehydration a composition described herein, wherein the administration of the composition rehydrates the skin, thereby treating skin dehydration.

In a fourth embodiment, the disclosure relates to a method of treating a lack of skin elasticity, comprising administering to an individual suffering from a lack of skin elasticity a composition described herein, wherein the administration of the composition increases the elasticity of the skin, thereby treating the lack of skin elasticity, skin paleness, skin wrinkles.

In a fifth embodiment, the disclosure relates to a method of treating skin roughness, comprising administering to an individual suffering from skin roughness a composition described herein, wherein the administration of the composition decreases skin roughness, thereby treating skin roughness.

In a sixth embodiment, the disclosure relates to a method of treating a lack of skin tautness, comprising administering to an individual suffering from a lack of skin tautness a composition described herein, wherein the administration of the composition makes the skin tauter, thereby treating the lack of skin tautness.

In a seventh embodiment, the disclosure relates to a method of treating a skin stretch line or mark, comprising administering to an individual suffering from a skin stretch line or mark a composition described herein, wherein the administration of the composition reduces or eliminates the skin stretch line or mark, thereby treating the skin stretch line or mark.

In eighth embodiment, the disclosure relates to a method of treating skin paleness, comprising administering to an individual suffering from skin paleness a composition described herein, wherein the administration of the composition increases skin tone or radiance, thereby treating skin paleness.

In a ninth embodiment, the disclosure relates to a method of treating skin wrinkles, comprising administering to an individual suffering from skin wrinkles a composition described herein, wherein the administration of the composition reduces or eliminates skin wrinkles, thereby treating skin wrinkles.

In a tenth embodiment, the disclosure relates to a method of treating skin wrinkles, comprising administering to an individual a composition disclosed herein, wherein the administration of the composition makes the skin resistant to skin wrinkles, thereby treating skin wrinkles.

In an eleventh embodiment, the disclosure relates to a method of preventing skin wrinkles, comprising administering to an individual a composition disclosed herein, wherein the administration of the composition makes the skin resistant to skin wrinkles, thereby preventing skin wrinkles.

In some embodiments, the administration is by subcutaneous injection.

In some embodiments, the administration occurs at a depth of less than about 1 mm below the surface of the skin.

In some embodiments, the method does not result in arterial occlusion.

In some embodiments, the method does not result in unpredictable augmentation.

In some embodiments, the method does not result in irritation, for example, chronic irritation.

In some embodiments, the composition is soluble in blood.

In some embodiments, the method results in limited swelling.

In some embodiments, the administration of the composition results in low immunogenicity.

In some embodiments, the disclosure relates to use of a hydrogel composition disclosed herein for subcutaneous injection.

In certain embodiments, the disclosure relates to a method of treating fine lines or superficial wrinkles in the skin of a subject, comprising administering a composition into a dermal region of the subject which displays the fine lines or superficial wrinkles, thereby treating the fine lines or superficial wrinkles, wherein the composition comprises a polypeptide hydrogel.

In some embodiments, the dermal region is a tear trough region, a glabellar line, a periorbital region, or a forehead region.

In another embodiment, the disclosure relates to a method of treating a skin condition, comprising administering to an individual suffering from the skin condition a composition, wherein the administration of the composition improves the skin condition, thereby treating the skin condition, wherein the composition comprises a polypeptide hydrogel.

In some embodiments, the skin condition is skin dehydration.

In some embodiments, the composition rehydrates the skin of the subject.

In some embodiments, the skin condition is skin elasticity.

In some embodiments, the composition increases the elasticity of the skin of the subject.

In some embodiments, the skin condition is skin roughness.

In some embodiments, the composition decreases skin roughness in the subject.

In some embodiments, the skin condition is a lack of skin tautness.

In some embodiments, the composition increases skin tautness in the subject.

In some embodiments, the skin condition is a skin stretch line or mark.

In some embodiments, the composition reduces or eliminates the skin stretch line or mark in the subject.

In some embodiments, the skin condition is skin paleness.

In some embodiments, the composition increases skin tone or radiance in the subject.

In some embodiments, the skin condition is skin wrinkles.

In some embodiments, the composition reduces or eliminates skin wrinkles in the subject.

In another embodiment, the disclosure relates to a method of preventing skin wrinkles in a subject, comprising administering to the subject a composition, thereby preventing skin wrinkles, wherein the composition comprises a polypeptide hydrogel.

In some embodiments, the composition increases or improves the resistance of the skin of the subject to skin wrinkles.

In some embodiments, the composition makes the skin of the subject resistant to skin wrinkles.

In some embodiments, the administration is by subcutaneous injection.

In some embodiments, the administration occurs at a depth of less than about 1 mm below the surface of the skin.

In some embodiments, the method does not result in arterial occlusion.

In some embodiments, the method does not result in unpredictable augmentation.

In some embodiments, the method does not result in irritation, for example, chronic irritation.

In some embodiments, the composition is soluble in blood.

In some embodiments, administration of the composition results in limited swelling.

In some embodiments, the administration of the composition results in low immunogenicity.

In some embodiments, the disclosure relates to any of the methods described herein, wherein the composition comprises a first copolypeptide comprising Substructure I, a second copolypeptide comprising Substructure II, and water,

wherein

Substructure I is depicted as follows:

—Xₘ—Cₚ— Substructure I;

Substructure II is depicted as follows:

—Yₙ-A_{q}- Substructure II;
each instance of X is an amino acid residue independently selected from a non-ionic, hydrophilic amino acid, glycine, alanine, and sarcosine;
each instance of Y is an amino acid residue independently selected from a non-ionic, hydrophilic amino acid, glycine, alanine, and sarcosine;
each instance of C is an amino acid residue independently selected from a cationic, hydrophilic amino acid;
each instance of A is an amino acid residue independently selected from an anionic, hydrophilic amino acid;
m is about 100 to about 600;
n is about 100 to about 600;
p is about 20 to about 200;
q is about 20 to about 200;
at least 90 mol % of the C amino acid residues are (D)-amino acid residues or at least 90 mol % of the C amino acid residues are (L)-amino acid residues; and
at least 90 mol % of the A amino acid residues are (D)-amino acid residues or at least 90 mol % of the A amino acid residues are (L)-amino acid residues.

In some embodiments, wherein
each instance of X is an amino acid residue independently selected from methionine sulfoxide (M^{o}), alanine (A), and sarcosine;
each instance of Y is an amino acid residue independently selected from methionine sulfoxide (M^{o}), alanine (A), and sarcosine;
each instance of C is the amino acid residue lysine (K); and
each instance of A is the amino acid residue glutamic acid (E).

In some embodiments, wherein
each instance of X is an amino acid residue independently selected from methionine sulfoxide (M^{o}) and alanine (A);
each instance of Y is an amino acid residue independently selected from methionine sulfoxide (M^{o}) and alanine (A);
each instance of C is the amino acid residue lysine (K); and
each instance of A is the amino acid residue glutamic acid (E). In some embodiments, about 88 mol % of the X amino acid residues are M^{o}, and about 12 mol % of the X amino acid residues are A; and
about 88 mol % of the Y amino acid residues are M^{O}, and about 12 mol % of the X amino acid residues are A.

In some embodiments, m is 155 or 180; and p is 55, 65, 75, or 85.

In some embodiments, n is 155 and q is 55, 65, 75, or 85.

In some embodiments, wherein

Substructure I is

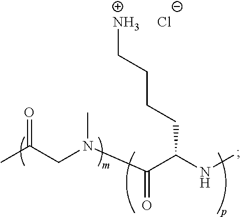

and

Substructure II is

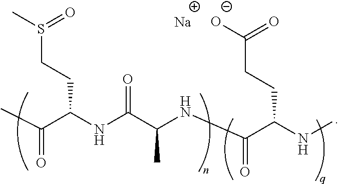

In some embodiments, Substructure I is (M^{O}A)₁₈₀-K₇₅; and Substructure II is (M^{O}A)₁₈₀-E₇₅.

In some embodiments, Substructure I is (M^{O}A)₁₅₅-K₅₅; and Substructure II is (M^{O}A)₁₅₅-E₅₅.

In some embodiments, Substructure I is (M^{O}A)₁₅₅-K₆₅; and Substructure II is (M^{O}A)₁₅₅-E₆₅.

In some embodiments, Substructure I is (M^{O}A)₁₅₅-K₇₅; and Substructure II is (M^{O}A)₁₅₅-E₇₅.

In some embodiments, Substructure I is (M^{O}A)₁₅₅-K₈₅; and Substructure II is (M^{O}A)₁₅₅-E₈₅.

In certain embodiments, wherein
each instance of X is the amino acid residue sarcosine;
each instance of Y is the amino acid residue sarcosine;
each instance of C is the amino acid residue lysine (K); and
each instance of A is the amino acid residue glutamic acid (E).

In some embodiments, wherein

Substructure I is

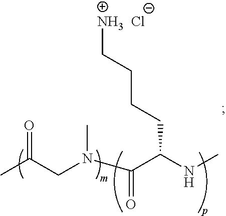

and

Substructure II is

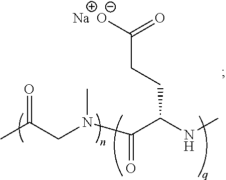

In some embodiments, Substructure I is (Sar)₁₅₀-K₆₅; and Substructure II is (Sar)₁₅₀-E₆₅.

In some embodiments, Substructure I is (Sar)₁₅₀-K₇₅; and Substructure II is (Sar)₁₅₀-E₆₅.

In some embodiments, Substructure I is (Sar)₁₅₀-K₆₅; and Substructure II is (Sar)₁₅₀-E₇₀.

In some embodiments, Substructure I is (Sar)₁₅₀-K₇₅; and Substructure II is (Sar)₁₅₀-E₇₀.

In some embodiments, the disclosure relates to any of the methods described herein, wherein the composition comprises a first copolypeptide comprising Substructure I, a second copolypeptide comprising Substructure II, and water,

wherein

Substructure I is depicted as follows:

—Cₚ—Xₘ— Substructure I;

Substructure II is depicted as follows:

-A_{q}-Yₙ— Substructure II;
each instance of X is an amino acid residue independently selected from a non-ionic, hydrophilic amino acid, glycine, alanine, and sarcosine;
each instance of Y is an amino acid residue independently selected from a non-ionic, hydrophilic amino acid, glycine, alanine, and sarcosine;
each instance of C is an amino acid residue independently selected from a cationic, hydrophilic amino acid;
each instance of A is an amino acid residue independently selected from an anionic, hydrophilic amino acid;
m is about 100 to about 600;
n is about 100 to about 600;
p is about 20 to about 200;
q is about 20 to about 200;
at least 90 mol % of the C amino acid residues are (D)-amino acid residues or at least 90 mol % of the C amino acid residues are (L)-amino acid residues; and
at least 90 mol % of the A amino acid residues are (D)-amino acid residues or at least 90 mol % of the A amino acid residues are (L)-amino acid residues.

In certain embodiments, wherein
each instance of X is the amino acid residue sarcosine;
each instance of Y is the amino acid residue sarcosine;
each instance of C is the amino acid residue lysine (K); and
each instance of A is the amino acid residue glutamic acid (E).

In some embodiments, wherein

Substructure I is

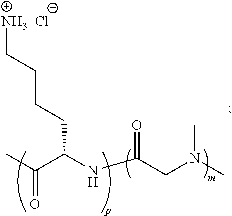

and

Substructure II is

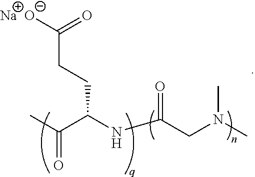

In some embodiments, Substructure I is K₆₅-(Sar)₁₅₀; and Substructure II is E₆₅-(Sar)₁₅₀.

In some embodiments, Substructure I is K₇₅-(Sar)₁₅₀; and Substructure II is E₆₅-(Sar)₁₅₀.

In some embodiments, Substructure I is K₆₅-(Sar)₁₅₀; and Substructure II is E₆₅-(Sar)₁₅₀.

In some embodiments, Substructure I is K₇₅-(Sar)₁₅₀; and Substructure II is E₆₅-(Sar)₁₅₀.

In some embodiments, m is 150; p is 65 or 70; n is 150 and q is 65 or 70.

In certain embodiments, the disclosure relates to any of the methods described herein, wherein an effective amount, such as a therapeutically effective amount or a prophylactically effective amount or a cosmetically effective amount, of the composition is administered.

### Compositions

I. In certain embodiments, the compositions used in the presently disclosed methods are described in and prepared by the methods disclosed in U.S. Pat. No. 8,691,204, which is hereby incorporated by reference in its entirety.
II. In certain embodiments, the compositions used in the presently disclosed methods are described in and prepared by the methods disclosed in U.S. Pat. No. 9,718,921, which is hereby incorporated by reference in its entirety.
III. In certain embodiments, the compositions used in the presently disclosed methods are described in and prepared by the methods disclosed in U.S. Patent Application Publication No. US2017/0296672, which is hereby incorporated by reference in its entirety.

In some aspects, the composition used in the disclosed methods comprises: an aqueous medium; and a copolypeptide hydrogel forming composition, wherein the copolypeptide composition comprises at least one hydrophilic polypeptide or copolypeptide segment and at least one hydrophobic polypeptide or copolypeptide segment, wherein the hydrophilic polypeptide or copolypeptide segment contains less than 50 mol % ionic amino acid residues.

In some embodiments, this composition can further comprise a second copolypeptide hydrogel forming composition, wherein said second copolypeptide composition comprises at least one hydrophilic polypeptide or copolypeptide segment and at least one thermoresponsive polypeptide or copolypeptide segment, wherein said second copolypeptide composition undergoes a temperature induced transition between a liquid and a transparent hydrogel in said aqueous medium.

In some embodiments, the composition comprising an aqueous medium; and a copolypeptide hydrogel forming composition, wherein said copolypeptide composition comprises at least one hydrophilic polypeptide or copolypeptide segment and at least one thermoresponsive polypeptide or copolypeptide segment, wherein said copolypeptide composition under goes a temperature induced transition between a liquid and a transparent hydrogel in said aqueous medium.

In certain embodiments, the copolypeptide hydrogels (DCH) contain less than 50 mol % ionic residues, i.e. either non-ionic (DCH_{EO}) or partially ionic. Some embodiments utilize poly(γ-[2-(2-methoxyethoxy)ethyl]-rac-glutamate), (rac-E_{P2}), as a hydrophilic segment.

In some embodiments of the compositions described above: (i) the sum of the lengths of all hydrophilic segments in a copolymer composition is between 120 and 600 residues, (ii) the sum of the lengths of all hydrophobic segments in a copolymer composition is between 20 and 100 residues, (iii) the copolymer contains 1 hydrophilic segment and 1 hydrophobic segment; (iv) the copolymer contains 2 hydrophilic segments and 1 hydrophobic segment; (v) amino acid residues in a hydrophobic segment may include leucine, alanine, phenylalanine, methionine, tyrosine, tryptophan, valine, isoleucine, serine, cysteine, glutamine, asparagine, γ-alkyl glutamate esters (e.g. γ-benzyl-glutamate), β-alkyl aspartate esters (e.g. β-benzyl-aspartate), ε-modified lysines (e.g. ε-trifluoroacetyl-lysine) and their mixtures; (vi) a hydrophobic segment possesses a predominantly α-helical conformation in water; (vii) non-ionic amino acid residues in a hydrophilic segment may include, but are not limited to, Non-ionic residues, and their mixtures; (viii) other amino acid residues in a hydrophilic segment, if present, may include, but are not limited to, lysine, glutamate, aspartate, arginine, ornithine, homoarginine, sulfonium derivatives of methionine, and their mixtures, or (ix) the entire copolypeptide in aqueous medium, at a concentration of <4 wt. %, forms a hydrogel, or any combination thereof. Individual partially ionic DCH or DCH_{EO} compositions may also be physically blended with other DCH_{EO} or ionic DCH compositions in any proportion, which allows fine tuning of the resulting hydrogel properties.

Examples of hydrogel forming non-ionic and partly ionic DCH_{EO} compositions are shown in Table 1 and Table 2 of U.S. Patent Application Publication No. US/2017/0296672.

In some embodiments, the composition used in the disclosed methods comprises: an aqueous medium; and
a copolypeptide hydrogel forming composition,
wherein said copolypeptide composition comprises at least one hydrophilic polypeptide segment or hydrophilic copolypeptide segment and at least one hydrophobic polypeptide segment or hydrophobic copolypeptide segment,
wherein the hydrophilic polypeptide segment or hydrophilic copolypeptide segment consists of residues selected from lysine, glutamate, aspartate, arginine, ornithine, homoarginine, a residue of Formula I, a residue of Formula II, a residue of Formula III, a residue of Formula IV, a residue of Formula V, a residue of Formula VI, and combinations thereof; and
wherein the hydrophilic polypeptide segment or hydrophilic copolypeptide segment contains less than 50 mol % ionic amino acid residues, wherein an ionic amino acid residue is an amino acid residue having a charged side-chain at pH=7 in water;

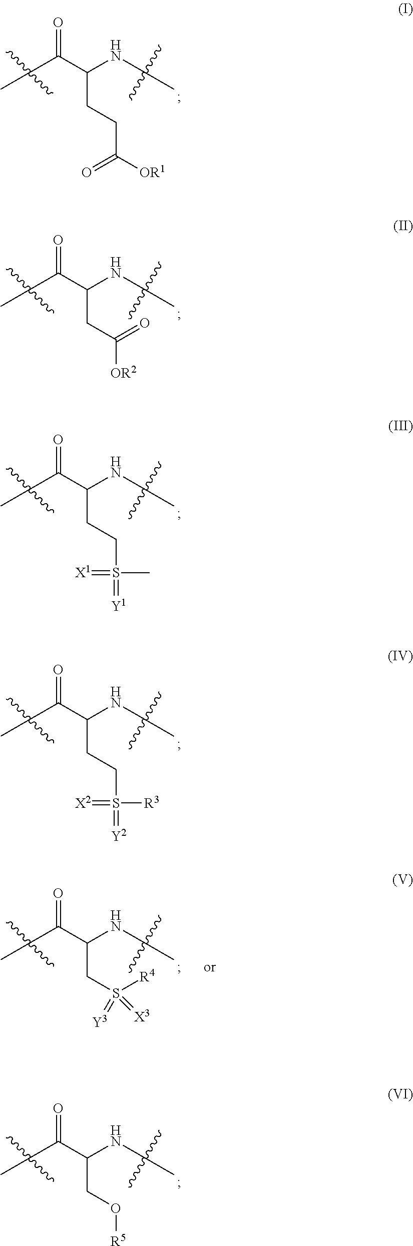

wherein:
R¹ is, independently at each occurrence, —(CH₂CH₂O)ₙCH₃ or

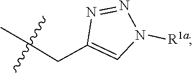

R^{1a} is —(CH₂CH₂O)ₙCH₃;
R² is, independently at each occurrence, —(CH₂CH₂O)ₙCH₃ or

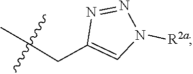

R^{2a} is —(CH₂CH₂O)ₙCH₃;
X¹ is O;
Y¹ is, independently at each occurrence, absent or O;
R³ is, independently at each occurrence, selected from —(CH₂CH₂O)ₘCH₃, —CH₂CH₂CH₂(sugar), and -sugar;
X² is, independently at each occurrence, absent or O;
Y² is, independently at each occurrence, absent or O;
R⁴ is, independently at each occurrence, selected from —(CH₂CH₂O)ₚCH₃, —CH₂CH₂CH₂(sugar), —CH₂CHR^{4a}C(O)OR^{4b}, and —CH₂CH₂SO₂CH₂CH₂SR^{4c};
R^{4a} is, independently at each occurrence, —H or —CH₃;
R^{4b} is —(CH₂CH₂O)ₚCH₃;
R^{4c} is —(CH₂CH₂O)ₚCH₃;
X³ is, independently at each occurrence, absent or O;
Y³ is, independently at each occurrence, absent or O;
R⁵ is, independently at each occurrence, —(CH₂CH₂O)˜CH₃ or -sugar;
n is an integer from 1-4;
m is an integer from 1-6; and
p is an integer from 1-9.

In some embodiments the composition further comprises an agent or a cell.

In some embodiments the composition further comprises a second copolypeptide hydrogel forming composition,
wherein said second copolypeptide composition comprises at least one hydrophilic polypeptide segment or hydrophilic copolypeptide segment and at least one thermoresponsive polypeptide segment or thermoresponsive copolypeptide segment,
wherein said second copolypeptide composition undergoes a temperature induced transition between a liquid and a transparent hydrogel in said aqueous medium.

In some embodiments the at least one thermoresponsive copolypeptide segment comprises at least one thermoresponsive residue and at least one non-ionic residue.

In some embodiments the composition further comprises an agent or a cell.

In some embodiments, a plurality of residues in the hydrophilic polypeptide segment or hydrophilic copolypeptide segment are selected from a residue of Formula I, a residue of Formula II, a residue of Formula III, a residue of Formula IV, a residue of Formula V, and a residue of Formula VI.

In some embodiments, the hydrophilic polypeptide segment or hydrophilic copolypeptide segment consists of residues selected from a residue of Formula I, a residue of Formula II, a residue of Formula III, a residue of Formula IV, a residue of Formula V, and a residue of Formula VI.

In some embodiments, the hydrophobic polypeptide segment or hydrophobic copolypeptide segment comprises residues selected from leucine, alanine, phenylalanine, methionine, tyrosine, tryptophan, valine, isoleucine, serine, cysteine, glutamine, asparagine, a γ-alkyl glutamate ester, a β-alkyl aspartate ester, and a ε-modified lysine.

In some embodiments, the copolypeptide is selected from:

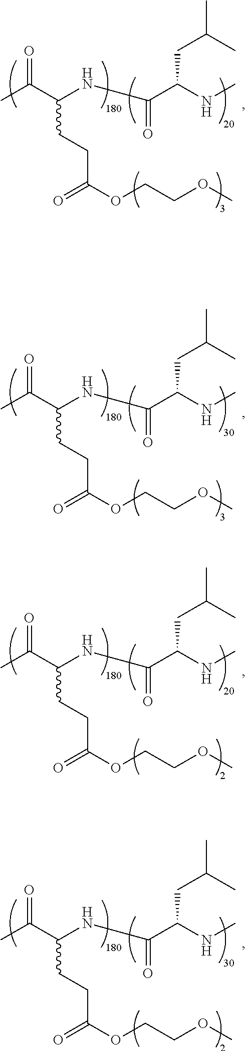

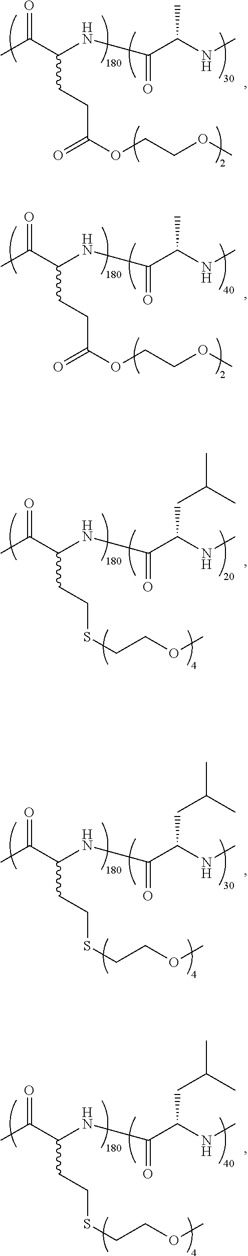

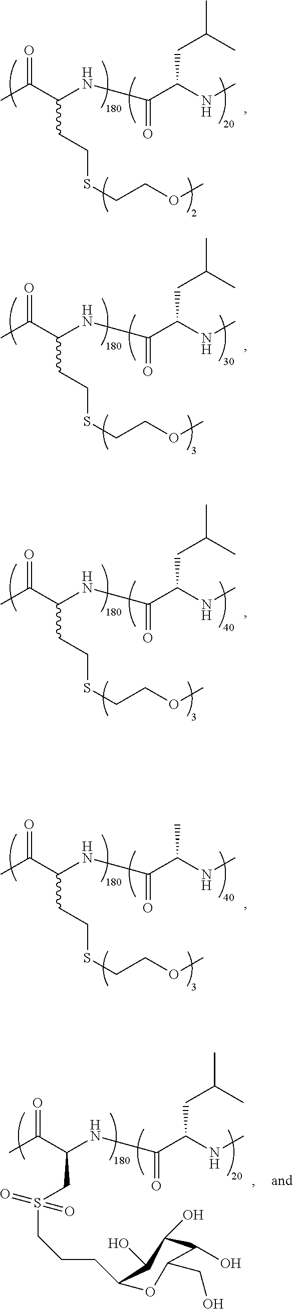

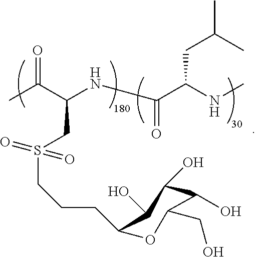

and

In some embodiments, the copolypeptide contains less than 50 mol % ionic amino acid residues.

In some embodiments, after exposure of a suspension of HeLa cells to the composition at a concentration of 2% for 24 hours, greater than 71% of the HeLa cells are viable.

In some embodiments, the sugar is selected from galactose, glucose, and mannose.
IV. In certain embodiments, the compositions used in the presently disclosed methods are described in and prepared by the methods disclosed in U.S. Patent Application Publication No. US/2019/0119322, which is hereby incorporated by reference in its entirety.
V. In certain embodiments, the compositions used in the presently disclosed methods are disclosed in and prepared by the methods disclosed in U.S. Patent Application Publication No. US2020/0246503, which is hereby incorporated by reference in its entirety.

In some aspects, the composition comprises a first copolypeptide comprising, consisting essentially of, or consisting of Substructure I, a second copolypeptide comprising, consisting essentially of, or consisting of Substructure II, and water,

wherein

Substructure I is depicted as follows:

—Xₘ—Cₚ— or —Cₚ—Xₘ— Substructure I;

Substructure II is depicted as follows:

—Yₙ-A_{q}- or-A_{q}-Yₙ— Substructure II;
each instance of X is an amino acid residue independently selected from a non-ionic, hydrophilic amino acid, glycine, alanine, and sarcosine;
each instance of Y is an amino acid residue independently selected from a non-ionic, hydrophilic amino acid, glycine, alanine, and sarcosine;
each instance of C is an amino acid residue independently selected from a cationic, hydrophilic amino acid;
each instance of A is an amino acid residue independently selected from an anionic, hydrophilic amino acid;
m is about 100 to about 600;
n is about 100 to about 600;
p is about 20 to about 200;
q is about 20 to about 200;
at least 90 mol % of the C amino acid residues are (D)-amino acid residues or at least 90 mol % of the C amino acid residues are (L)-amino acid residues; and
at least 90 mol % of the A amino acid residues are (D)-amino acid residues or at least 90 mol % of the A amino acid residues are (L)-amino acid residues.

In certain embodiments, the first copolypeptide comprises only amino acid residues. In certain embodiments, the second copolypeptide comprises only amino acid residues. In certain embodiments, the first copolypeptide and the second copolypeptide comprise only amino acid residues.

In certain embodiments, the first copolypeptide does not comprise PEG. In certain embodiments, the second copolypeptide does not comprise PEG. In certain embodiments, the first copolypeptide and the second copolypeptide do not comprise PEG.

In certain embodiments, the first copolypeptide is a diblock copolypeptide. In certain embodiments, the second copolypeptide is a diblock copolypeptide. In certain embodiments, the first copolypeptide and the second copolypeptide are diblock copolypeptides.

In certain embodiments, —Xₘ— has a primarily disordered configuration, for example, a configuration that is less than about 20% helical or less than about 20% beta-sheet.

In certain embodiments, —Yₙ— has a primarily disordered configuration, for example, a configuration that is less than about 20% helical or less than about 20% beta-sheet.

In certain embodiments, each instance of X is an amino acid residue independently selected from methionine sulfoxide, S-alkyl-cysteine sulfoxide, S-alkyl cysteine sulfone, glycosylated cysteine, serine, homoserine, homomethionine sulfoxide, glycine, alanine, and sarcosine.

In certain embodiments, each instance of Y is an amino acid residue independently selected from methionine sulfoxide, S-alkyl-cysteine sulfoxide, S-alkyl cysteine sulfone, glycosylated cysteine, serine, homoserine, homomethionine sulfoxide, glycine, alanine, and sarcosine.

In certain aspects, the composition comprises a first copolypeptide comprising Substructure I, a second copolypeptide comprising Substructure II, and water, wherein

Substructure I is depicted as follows:

—Xₘ—Cₚ— or —Cₚ—Xₘ— Substructure I;

Substructure II is depicted as follows:

—Yₙ-A_{q}- or-A_{q}-Yₙ— Substructure II;
each instance of X is an amino acid residue independently selected from methionine sulfoxide, S-alkyl-cysteine sulfoxide, S-alkyl cysteine sulfone, glycosylated cysteine, serine, homoserine, homomethionine sulfoxide, glycine, and alanine;
each instance of Y is an amino acid residue independently selected from methionine sulfoxide, S-alkyl-cysteine sulfoxide, S-alkyl cysteine sulfone, glycosylated cysteine, serine, homoserine, homomethionine sulfoxide, glycine, and alanine;
each instance of C is an amino acid residue independently selected from lysine and arginine;
each instance of A is an amino acid residue independently selected from glutamic acid and aspartic acid;
m is about 100 to about 600;
n is about 100 to about 600;
p is about 20 to about 200;
q is about 20 to about 200;
at least 90 mol % of the C amino acid residues are (D)-amino acid residues or at least 90 mol % of the C amino acid residues are (L)-amino acid residues; and
at least 90 mol % of the A amino acid residues are (D)-amino acid residues or at least 90 mol % of the A amino acid residues are (L)-amino acid residues.

In certain embodiments, the first copolypeptide, the second copolypeptide, and the water are in admixture.

In certain embodiments, —Xₘ— has a primarily disordered configuration, for example, a configuration that is less than about 20% helical or less than about 20% beta-sheet.

In certain embodiments, —Yₙ— has a primarily disordered configuration, for example, a configuration that is less than about 20% helical or less than about 20% beta-sheet.

In certain embodiments, at least 80 mol % of the X amino acid residues are a sulfur-containing amino acid.

In certain embodiments, at least 80 mol % of the Y amino acid residues are a sulfur-containing amino acid.

In certain embodiments, at least 80 mol % of the X amino acid residues are methionine sulfoxide.

In certain embodiments, at least 90 mol % of the X amino acid residues are (D)-amino acid residues or at least 90 mol % of the X amino acid residues are (L)-amino acid residues.

In certain embodiments, at least 90 mol % of the X amino acid residues are (D)-amino acid residues.

In certain embodiments, at least 90 mol % of the X amino acid residues are (L)-amino acid residues.

In certain embodiments, at least 85 mol % of the X amino acid residues are methionine sulfoxide.

In certain embodiments, at least 85 mol % of the X amino acid residues are methionine sulfoxide, and the remaining X amino acid residues are alanine.

In certain embodiments, about 88 mol % of the X amino acid residues are methionine sulfoxide, and about 12 mol % of the X amino acid residues are alanine.

In certain embodiments, at least 80 mol % of the Y amino acid residues are methionine sulfoxide.

In certain embodiments, at least 90 mol % of the Y amino acid residues are (D)-amino acid residues or at least 90% of the Y amino acid residues are (L)-amino acid residues.

In certain embodiments, at least 90 mol % of the Y amino acid residues are (D)-amino acid residues.

In certain embodiments, at least 90% of the Y amino acid residues are (L)-amino acid residues.

In certain embodiments, at least 85 mol % of the Y amino acid residues are methionine sulfoxide.

In certain embodiments, at least 85 mol % of the Y amino acid residues are methionine sulfoxide, and the remaining Y amino acid residues are alanine.

In certain embodiments, about 88 mol % of the Y amino acid residues are methionine sulfoxide, and about 12 mol % of the Y amino acid residues are alanine.

In certain embodiments, at least 90% of the C amino acid residues are (D)-amino acid residues.

In certain embodiments, at least 90% of the C amino acid residues are (L)-amino acid residues.

In certain embodiments, each instance of C is lysine.

In certain embodiments, each instance of C is (L)-lysine.

In certain embodiments each instance of C is (D)-lysine.

In certain embodiments, at least 90% of the A amino acid residues are (D)-amino acid residues.

In certain embodiments, at least 90% of the A amino acid residues are (L)-amino acid residues.

In certain embodiments, each instance of A is glutamic acid.

In certain embodiments, each instance of A is (L)-glutamic acid.

In certain embodiments, each instance of A is (D)-glutamic acid.

In certain embodiments, m is about 100, about 110, about 120, about 130, about 140, about 150, about 160, about 170, about 180, about 190, about 200, about 210, or about 220.

In certain embodiments, m is about 120, about 130, about 140, about 150, about 160, about 170, about 180, or about 190.

In certain embodiments, n is about 100, about 110, about 120, about 130, about 140, about 150, about 160, about 170, about 180, about 190, about 200, about 210, or about 220.

In certain embodiments, n is about 120, about 130, about 140, about 150, about 160, about 170, about 180, or about 190.

In certain embodiments, p is about 20, about 30, about 40, about 50, about 60, about 70, about 80, about 90, about 100, about 110, about 120, or about 130.

In certain embodiments, q is about 20, about 30, about 40, about 50, about 60, about 70, about 80, about 90, about 100, about 110, about 120, or about 130.

In certain embodiments, the polydispersity of the first copolypeptide is less than 1.5.

In certain embodiments, the polydispersity of the second copolypeptide is less than 1.5.

In certain embodiments, the number of amino acid residues in the first copolypeptide is from about 90% to about 110% of the number of amino acid residues in the second copolypeptide.

In certain embodiments, the total concentration of the first copolypeptide and the second copolypeptide in the composition is greater than about 2.0 wt. %.

In certain embodiments, the total concentration of the first copolypeptide and the second copolypeptide in the composition is greater than about 3.0 wt. %.

In certain embodiments, the total concentration of the first copolypeptide and the second copolypeptide in the composition is greater than about 4.0 wt. %.

In certain embodiments, the total concentration of the first copolypeptide and the second copolypeptide in the composition is about 5.0 wt. %

In certain embodiments, the molar ratio of C to A is from about 0.95 to about 1.05.

In certain embodiments, the molar ratio of X to Y is from about 0.95 to about 1.05.

In certain embodiments, the composition further comprises a salt.

In certain embodiments, the concentration of the salt in the composition is less than about 500 mM.

In certain embodiments, the concentration of the salt in the composition is from about 100 mM to about 300 mM.

In certain embodiments, the salt is NaCl.

In certain embodiments, the composition further comprises a buffer.

In some embodiments, the composition comprises (M^{O}A)₁₅₅E₃₀, (M^{O}A)₁₅₅E₆₀, (M^{O}A)₁₅₅E₉₀, (M^{O}A)₁₅₅E₁₂₀, (M^{O}A)₁₅₅(rac-E)₆₀, (M^{O}A)₁₅₅K₃₀, (M^{O}A)₁₅₅K₆₀, (M^{O}A)₁₅₅K₉₀, or (M^{O}A)₁₅₅K₁₂₀.

In some embodiments, the composition comprises (M^{O}A)₁₅₅E₃₀.

In some embodiments, the composition comprises (M^{O}A)₁₅₅E₆₀.

In some embodiments, the composition comprises (M^{O}A)₁₅₅E₉₀.

In some embodiments, the composition comprises (M^{O}A)₁₅₅E₁₂₀.

In some embodiments, the composition comprises (M^{O}A)₁₅₅(rac-E)₆₀.

In some embodiments, the composition comprises (M^{O}A)₁₅₅K₃₀.

In some embodiments, the composition comprises (M^{O}A)₁₅₅K₆₀.

In some embodiments, the composition comprises (M^{O}A)₁₅₅K₉₀.

In some embodiments, the composition comprises (M^{O}A)₁₅₅K₁₂₀.

In certain embodiments, the composition further comprises a plurality of cells.

In certain embodiments of the compositions used in the disclosed methods
each instance of X is an amino acid residue independently selected from methionine sulfoxide and alanine;
each instance of Y is an amino acid residue independently selected from methionine sulfoxide and alanine;
each instance of C is the amino acid residue lysine; and
each instance of A is the amino acid residue glutamic acid.

In some embodiments, the composition comprises (M^{O}A)₁₅₅E₆₅.

In some embodiments, the composition comprises (M^{O}A)₁₅₅E₇₅

In some embodiments, the composition comprises (M^{O}A)₁₅₅E₈₅.

In some embodiments, the composition comprises (M^{O}A)₁₅₅E₇₅.

In some embodiments, the composition comprises (M^{O}A)₁₅₅K₆₅.

In some embodiments, the composition comprises (M^{O}A)₁₅₅K₇₅

In some embodiments, the composition comprises (M^{O}A)₁₅₅K₈₅.

In some embodiments, the composition comprises (M^{O}A)₁₈₀K₇₅.

In some embodiments, the composition further comprises a local anesthetic. In some embodiments, the composition further comprises lidocaine. In some embodiments, the lidocaine is present at 0.1-0.5% w/w of the composition. In some embodiments, the lidocaine is present at about 0.3% w/w of the composition.

### Synthesis and Characterization of Above Copolypeptides Comprising Substructure I and Substructure II:

### Materials and Instrumentation.

Tetrahydrofuran (THF), hexanes, and methylene chloride were dried by purging with nitrogen and passage through activated alumina columns prior to use. Co(PMe₃)₄ and amino acid N-carboxyanhydride (NCA) monomers were prepared according to literature procedures. Kramer, J. R.; Deming, T. J. Biomacromolecules 2012, 13, 1719-1723. All other chemicals were purchased from commercial suppliers and used without further purification unless otherwise noted. Selecto silica gel 60 (particle size 0.032-0.063 mm) was used for flash column chromatography. Fourier Transform Infrared (FTIR) measurements were taken on a Perkin Elmer RX1 FTIR spectrophotometer calibrated using polystyrene film, and attenuated total reflectance (ATR-IR) data were collected using a PerkinElmer Spectrum 100 FTIR spectrometer equipped with a universal ATR sample accessory. ¹H NMR spectra were acquired on a Bruker ARX 400 spectrometer. Tandem gel permeation chromatography/light scattering (GPC/LS) was performed at 25° C. using an SSI Accuflow Series III pump equipped with Wyatt DAWN EOS light scattering and Optilab REX refractive index detectors. Separations were achieved using 100 Å and 1000 Å PSS-PFG 7 μm columns at 30° C. with 0.5% (w/w) KTFA in 1,1,1,3,3,3-hexafluoroisopropanol (HFIP) as eluent and sample concentrations of 10 mg/ml. Pyrogen free deionized water (DI) was obtained from a Millipore Milli-Q Biocel A10 purification unit. Circular Dichroism spectra were recorded in quartz cuvettes of 0.1 cm path length with samples prepared at concentrations between 0.10 to 0.17 mg/mL using Millipore deionized water. The spectra are reported in units of molar ellipticity [θ](deg·cm²·dmol⁻¹), using the formula, [θ]=(θ×100×Mw)/(c×l), where θ is the measured ellipticity in millidegrees, Mw, is the average residue molecular mass in g/mol, c is the peptide concentration in mg/mL; and I is the cuvette path length in cm.

### General Procedure for Copolypeptide Preparation

All polymerization reactions were performed in an N₂ filled glove box using anhydrous solvents. To a solution of L-methionine NCA (Met NCA) and L-alanine NCA (Ala NCA) in THF (50 mg/ml), a solution of Co(PMe₃)₄ in THF (20 mg/ml) was added. The reaction was let to stir at ambient temperature (ca. 22° C.) for 60 min. Complete consumption of NCA was confirmed by FTIR spectroscopy, and then the desired amount of γ-benzyl-L-glutamate NCA (Bn-Glu NCA) or ε-TFA-L-lysine NCA (TFA-Lys NCA) in THF (50 mg/ml) was added to the reaction mixture, which was let to stir for an additional 60 min. FTIR was used to confirm complete consumption of all NCAs. Outside the glove box, the block copolypeptide solutions were precipitated into 10 mM HCl (20 ml), and then washed with 10 mM aqueous HCl (2×20 ml) to remove residual cobalt ions. The white precipitates were then washed with DI water (3×20 ml) and freeze-dried.

TABLE A
Copolymerization data for diblock copolypeptide synthesis.

| Sample | M_{w}/Mₙ^{a} | Composition^{b} | Yield (%)^{c} |
|---|---|---|---|
| (M^{o}A)₁₅₅E₃₀ | 1.35 | (M^{o}A)₁₅₆E₂₇ | 94 |
| (M^{o}A)₁₅₅E₆₀ | 1.41 | (M^{o}A)₁₅₆E₅₉ | 96 |
| (M^{o}A)₁₅₅E₉₀ | 1.45 | (M^{o}A)₁₅₆E₈₈ | 92 |
| (M^{o}A)₁₅₅E₁₂₀ | 1.42 | (M^{o}A)₁₅₆E₁₁₇ | 97 |
| (M^{o}A)₁₅₅(rac-E)₆₀ | 1.45 | (M^{o}A)₁₅₆(rac-E)₅₆ | 92 |
| (M^{o}A)₁₅₅K₃₀ | 1.38 | (M^{o}A)₁₅₆K₂₈ | 97 |
| (M^{o}A)₁₅₅K₆₀ | 1.41 | (M^{o}A)₁₅₆K₆₂ | 95 |
| (M^{o}A)₁₅₅K₉₀ | 1.40 | (M^{o}A)₁₅₆K₈₈ | 95 |
| (M^{o}A)₁₅₅K₁₂₀ | 1.37 | (M^{o}A)₁₅₆K₁₁₉ | 96 |

^{a}Dispersity of oxidized, protected block copolypeptides were determined by GPC/LS.
^{b}Relative amino acid compositions of oxidized, deprotected block copolypeptides were determined by ¹H NMR integrations. Degree of polymerization of initial MAₓ segment was determined by end-group analysis using ¹H NMR.
^{c}Total isolated yield of purified block copolypeptides following deprotection.

### Example synthesis of poly(L-methionine_{0.88}-stat-L-alanine_{0.12})₁₅₅-block-poly(ε-trifluoroacetyl-L-lysine)₆₀, (MA)₁₅₅(TFA-K)₅₅ and poly(L-methionine_{0.88}-stat-L-alanine_{0.12})₁₅₅-block-poly(γ-benzyl-L-glutamate)₆₀, (MA)₁₅₅(Bn-E)₆₀

Met NCA (120 mg, 0.71 mmol) and Ala NCA (11 mg, 0.097 mmol) were dissolved together in THF (2.7 ml) and placed in a 20 ml scintillation vial containing a stir bar. To the vial, (PMe₃)₄Co initiator solution (260 μl of a 20 mg/ml solution in THF) was added via syringe. The vial was sealed and allowed to stir in the glove box for 1 h. An aliquot (20 μl) was removed and analyzed by FTIR to confirm that all the NCA was consumed. In the glove box, α-methoxy-ω-isocyanoethyl-poly(ethylene glycol)₄₅ (mPEG₂₃-NCO) (20 mg) was dissolved in THF (1 ml) in a 20 ml scintillation vial. An aliquot (350 μl) of the polymerization solution containing active chain ends was removed and added to the solution of mPEG₂₃-NCO. The PEG end-capped sample (MAX-mPEG₂₃) was sealed, allowed to stir for 24 h, and then used for chain length determination (vide infra). Separately, aliquots of the polymerization solution containing active chains (1.2 ml each) were added to vials containing either Bn-Glu NCA (32 mg, 0.12 mmol) or TFA-Lys NCA (33 mg, 0.12 mmol) dissolved in THF (64 μl or 65 μl, respectively). The vials were sealed and allowed to stir in the glove box for 1 h to give the diblock copolypeptides, (MA)₁₅₅(TFA-K)₆₀ and (MA)₁₅₅(Bn-E)₆₀.FTIR was used to confirm complete consumption of NCAs in both reactions. Outside the glove box, the block copolypeptide solutions were precipitated into 10 mM HCl (20 ml), and then washed with 10 mM aqueous HCl (2×20 ml) to remove residual cobalt ions. The white precipitates were then washed with DI water (3×20 ml) and freeze-dried (average yield=98%).

Analytical data: (MA)₁₅₅(Bn-E)₆₀

¹H NMR (400 MHz, d-TFA, 25° C.): δ 7.38 (br m, 2.3H), 5.24 (br m, 0.93H), 4.97 (br s, 1H), 4.81 (br m, 0.54H), 2.81 (br m, 2H), 2.6 (br m, 1.06 H), 2.40-2.05 (br m, 6.37H), 1.61 (br s, 0.42H). FTIR (THF, 25° C.): 1738 cm⁻¹ (benzyl ester), 1652 cm⁻¹ (amide I), 1550 cm⁻¹ (amide II).

Analytical data: (MA)₁₅₅(TFA-K)₆₀

¹H NMR (400 MHz, d-TFA, 25° C.): δ 4.86 (br s, 0.94 H), 4.60 (br m, 0.54H), 3.46 (br m, 1.23 H), 2.69 (br m, 2H), 2.17 (br m, 5H), 1.9 (br m, 1.42 H), 1.69 (br m, 1.34 H), 1.50 (br m, 1.32 H), 1.31 (br m, 0.68 H). FTIR (THF, 25° C.): 1726 cm⁻¹ (TFA amide), 1652 cm⁻¹ (amide I), 1550 cm⁻¹ (amide II).

### Sample Procedure for MAₓ Chain Length Determination Using End-Group Analysis

Outside of the glove box, the PEG end-capped sample (MAₓ-mPEG₂₃) from above was washed with 10 mM aqueous HCl (2×). After stirring for 1 h, MAₓ-mPEG₂₃ was collected by centrifugation and washed with DI water (3×20 ml) to remove all non-conjugated mPEG₂₃-NCO. The remaining MAₓ-mPEG₂₃ was then freeze-dried to remove residual H₂O. To determine MAₓ molecular weights (Mₙ), ¹H NMR spectra were obtained. Since it has been shown that end-capping is quantitative for (PMe₃)₄Co initiated NCA polymerizations when excess isocyanate is used, integrations of methionine (δ 2.70) and alanine (δ 1.52) resonances versus the polyethylene glycol resonance at δ 3.92 could be used to obtain both M to A ratios and MAₓ lengths (found: x=156, designated as MA₁₅₅). ¹H NMR (400 MHz, d-TFA, 25° C.): 4.87 (br s, 1H), 4.68 (br s, 0.167H), 3.92 (br m, 0.71H), 2.70 (br m, 2.03 H), 2.30-2.05 (br m, 5.16H), 1.52 (br s, 0.43H).

### Preparation of poly(L-methionine sulfoxide_{0.88}-stat-L-alanine_{0.12})₁₅₅-block-poly(ε-trifluoroacetyl-L-lysine)₆₀, (M^{O}A)₁₅₅(TFA-K)₆₀, and poly(L-methionine sulfoxide_{0.88}-stat-L-alanine_{0.12})₁₅₅-block-poly(γ-benzyl-L-glutamate)₆₀, (M^{O}A)₁₅₅(Bn-E)₆₀

In separate scintillation vials (5 ml) containing stir bars, (MA)₁₅₅(TFA-K)₆₀ and (MA)₁₅₅(Bn-E)₆₀ were suspended in 80% tert-butyl hydroperoxide (TBHP) in water (16 eq TBHP per methionine residue). Camphorsulfonic acid (0.2 eq per methionine residue) was then added to each vial, and DI water was added to give final copolymer concentrations of ca. 20 mg/ml. These reactions were stirred for 16 h at ambient temperature (ca. 22° C.). Saturated sodium thiosulfate (0.5 ml) was then added dropwise to each vial in order to quench the reactions, and the samples were transferred to 2000 MWCO dialysis tubes and then dialyzed against DI water for 2 d with frequent water changes. The resulting solutions were freeze-dried to yield white fluffy solids (average yield=97%).

Analytical Data: (M^{O}A)₁₅₅(Bn-E)₆₀

¹H NMR (400 MHz, d-TFA, 25° C.): δ 7.24 (br m, 2.2H), 5.10 (br m, 0.91H), 4.85 (br s, 1H), 4.69 (br m, 0.55H), 3.45-3.10 (br m, 2.06H), 2.90 (br m, 3H), 2.62 (br m, 1.04 H), 2.47 (br m, 1.86 H), 2.18 (br m, 0.45H), 1.97 (br m, 0.45), 1.49 (br s, 0.40 H).

Analytical Data: (M^{O}A)₁₅₅(TFA-K)₆₀

¹H NMR (400 MHz, d-TFA, 25° C.): δ 4.91 (br s, 1H), 4.64 (br m, 0.52H), 3.52-3.10 (br m, 2.96 H), 2.96 (br m, 3.03H), 2.67 (br m, 1.04 H), 2.46 (br m, 1H), 1.96 (br m, 0.86 H), 1.73 (br m, 0.88 H), 1.54 (br m, 1.27 H).

### Preparation of poly(L-methionine sulfoxide_{0.88}-stat-L-alanine_{0.12})₁₅₅-block-poly(L-lysine)₆₀, (M^{O}A)₁₅₅K₆₀

A sample of (M^{O}A)₁₅₅(TFA-K)₆₀ was dispersed in a 9:1 methanol:water mixture (20 mg/ml) and K₂CO₃ (2 eq per lysine residue) was added. The reaction was stirred for 8 h at 50° C., and the majority of the methanol was then removed under vacuum. The resulting solution (ca. 10% of original volume) was then diluted with water (3 times the remaining volume), transferred to a 2000 MWCO dialysis bag, and then dialyzed against 0.10 M aqueous NaCl at pH 3 (HCl) for 24 h, followed by DI water for 48 hours with water changes twice per day. The contents of the dialysis bag were then lyophilized to dryness to give a white solid (yield=93%).^{1 1}H NMR (400 MHz, D₂O, 25° C.): δ 4.52 (br s, 1H), 4.37 (br m, 0.52H), 3.2-2.8 (br m, 3.18 H), 2.75 (br m, 3.1 H), 2.40-2.20 (br m, 2.2 H), 1.73 (br m, 1.62H), 1.44 (br m, 1.32H). ATR-IR (25° C.): 1653 cm⁻¹ (amide I), 1546 cm⁻¹ (amide II).

### Preparation of poly(L-methionine sulfoxide_{0.88}-stat-L-alanine_{0.12})₁₅₅-block-poly(L-glutamate)₆₀, (M^{O}A)₁₅₅E₆₀

A sample of (M^{O}A)₁₅₅(Bn-E)₆₀ was dissolved in trifluoroacetic acid (TFA, 30 eq per benzyl glutamate residue) in an ice bath. Methanesulfonic acid (MSA, 35 eq) and anisole (5 eq) were then added under vigorous stirring. The reaction mixture was stirred for 20 min in the ice bath, and then for an additional 90 min at ambient temperature. Next, the copolymer was precipitated using Et₂O (20 ml) and collected by centrifugation. The pellet was dissolved in 10% aqueous NaHCO₃ (3 ml), extensively dialyzed (2000 MWCO) against DI water for 2 d, and then freeze-dried to give a white solid (yield=95%).^{4 1}H NMR (400 MHz, D₂O, 25° C.): δ 4.50 (br s, 1H), 4.40 (br m, 0.57H), 3.00 (br m, 2.03H), 2.75 (br m, 2.95 H), 2.40-2.10 (br m, 3H), 2.10-1.80 (br m, 1H), 1.44 (br s, 0.4 H). ATR-IR (25° C.): 1653 cm⁻¹ (amide I), 1546 cm⁻¹ (amide II).

### Example synthesis of poly(L-methionine sulfoxide_{0.90}-stat-L-alanine_{0.10})₉₈, (M^{O}/_{0.90}A/_{0.10})₉₈, test copolymer

Met NCA (50 mg, 0.29 mmol) and Ala NCA (3.3 mg, 0.029 mmol) were dissolved together in THF (50 mg/mL) and placed in a 20 ml scintillation vial containing a stir bar. To the vial, (PMe₃)₄Co initiator solution (140 μl of a 20 mg/ml solution in THF) was added via syringe. The vial was sealed and allowed to stir in the glove box for 1 h. An aliquot (20 μl) was removed and analyzed by FTIR to confirm that all the NCA was consumed. In the glove box, mPEG₂₃-NCO (20 mg) was dissolved in THF (1 ml) in a 20 ml scintillation vial. An aliquot (350 μl) of the polymerization solution containing active chain ends was removed and added to the solution of mPEG₂₃-NCO. The PEG end-capped sample was sealed, allowed to stir for 24 h, and oxidized to give the methionine sulfoxide derivative, (M^{O}/_{0.90}A/_{0.10})₉₈-mPEG₂₃, which was then used for chain length determination as described above. The remainder of the polymerization mixture was isolated by precipitation, and then oxidized to the product methionine sulfoxide derivative, (M^{O}/_{0.90}A/_{0.10})₉₈, following standard procedures described above. Copolymers with different M to A ratios were prepared following similar procedures.

Preparation of (M^{O}A)₁₅₅E/Kₓ PIC hydrogels

Samples of (M^{O}A)₁₅₅Ex and (M^{O}A)₁₅₅Kₓ were separately dissolved in a desired aqueous medium (e.g. DI water, 1× PBS, etc.) at a desired concentration (e.g. 2.0, 3.0, or 5.0 wt %). Once each copolymer was fully dissolved, equal volumes of the copolymer solutions were combined in a scintillation vial (2 ml) and vortexed rigorously for 15 s using a Fisher Vortex Genie 2. The concentration of PIC hydrogel was defined as the sum of the concentrations of the two components after mixing, which is essentially the same as the starting concentrations of each component before mixing. The duration of time before gelation occurred (i.e. gelation time) was found to vary from seconds to minutes depending on sample concentration, the ionic strength, and copolymer composition. A “5 second inversion test” was used to initially confirm gel formation. Zhang, S. et al. Biomacromolecules 2015, 16, 1331-1340.

Rheology Measurements on (M^{O}A)₁₅₅E/Kₓ PIC Hydrogels

A TA Instruments AR 2000 rheometer with a 20 mm parallel plate geometry and solvent trap was used for all measurements. Frequency sweeps were measured at a constant strain amplitude of 0.05. Strain sweeps were measured at a constant frequency of 5 rad/s. All measurements were performed in the linear regime and were repeated 3 times for each hydrogel sample and the results were averaged and plotted.

TABLE B
Properties of diblock copolypeptide PIC hydrogels.

| Sample | Concentration (wt %) | G′ (Pa) | G″ (Pa) | Clarity |
|---|---|---|---|---|
| (M^{o}A)₁₅₅E/K₃₀ | 5.0 | 30 | 4 | translucent |
| (M^{o}A)₁₅₅E/K₉₀ | 5.0 | 99 | 7 | opaque |
| (M^{o}A)₁₅₅E/K₁₂₀ | 5.0 | 197 | 15 | opaque |
| (M^{o}A)₁₅₅E/K₆₀ | 2.0 | 3 | 0.7 | translucent |
| (M^{o}A)₁₅₅E/K₆₀ | 3.0 | 29 | 2 | translucent |
| (M^{o}A)₁₅₅E/K₆₀ | 5.0 | 116 | 9 | translucent |
| (M^{o}A)₁₅₅E/K₆₀ | 7.0 | 484 | 22 | translucent |
| (M^{o}A)₁₅₅E/K₆₀ | 15 | 2280 | 181 | translucent |

Samples were prepared in PBS buffer, 20° C. G′ = storage modulus; G″ = loss modulus.

Values are averages of triplicate runs at 5 rad/s and strain amplitude of 0.05. In general, the standard errors for frequency sweeps were less than 3.5%, while the standard errors for strain sweeps were less than 2.5%.
Fluorescent probe conjugation to (M^{O}A)₁₅₅E₆₀ and (M^{O}A)₁₅₅K₆₀ copolypeptides

Tetramethylrhodamine isothiocyanate (TRITC) was conjugated to amine groups of lysine side chains. (M^{O}A)₁₅₅K₆₀ (10 mg) was dissolved in pH 10 H₂O/NaOH (1 ml) in a scintillation vial (20 ml). TRITC was dissolved in DMSO (1 mg/ml) and added to the 1% (w/v) copolypeptide solution at a 5:1 molar ratio of copolypeptide chains to fluorescent probes. The reaction was allowed to proceed for 24 h at ambient temperature. After TRITC modification, the resulting solution was dialyzed (2000 MWCO) against DI water for 2 d, and then freeze-dried to yield the product as an orange solid. Fluorescein isothiocyanate (FITC) was conjugated onto the N-terminal amine of (M^{O}A)₁₅₅E₆₀ using a similar procedure.

### Laser Scanning Confocal Microscopy (LSCM) of Fluorescently Labeled Hydrogels

LSCM images of hydrogels (3.0 wt % in PBS) were taken on a Leica TCS-SP1 MP-Inverted Confocal and Multiphoton Microscope equipped with an argon laser (476 and 488 nm blue lines), a diode (DPSS) laser (561 nm yellow-green line), and a helium-neon laser (633 nm far red line). Fluorescently labeled hydrogel samples were visualized on glass slides with a spacer between the slide and the cover slip (double-sided tape) allowing the self-assembled structures to be minimally disturbed during focusing. A Z-slice thickness of 0.78 μm was used. Sample imaging was performed at the Advanced Light Microscopy/Spectroscopy Center (ALMS) at the UCLA California NanoSystems Institute (CNSI).

Cryoelectron Microscopy (cryoEM) of Hydrogels

25 μl of a 2.0 wt % (M^{O}A)₁₅₅E/K₆₀ hydrogel in PBS buffer was applied on a glass coverslip to form a flat surface onto which a lacey carbon EM grid was gently placed using a pair of tweezers in order to acquire a thin layer of sample. The EM grid was then plunged into liquid nitrogen-cooled ethane to prepare the grid for cryoEM. The vitrified sample was examined in an FEI TF20 cryoelectron microscope at liquid nitrogen temperature. Low dose cryoEM images were recorded on a CCD camera at 4.4 Å/pixel on the specimen level and a defocus value of about −5 μm. Sample preparation and imaging was performed at the Electron Imaging Center for Nanomachines (EICN) at the UCLA California NanoSystems Institute (CNSI).

### Viability of Neural Stem Progenitor Cells (NSPCs) Encapsulated in Hydrogels

NSPCs were harvested from the brain cortex of postnatal day 2 (P2) mice using procedures described in detail previously. Zhang, S. et al. ACS Biomater. Sci. Eng. 2015, 1, 705-717. Tissues around the ventricles were excised, diced with a razor blade and placed in Accumax solution (Innovative Cell Technologies, San Diego, CA) for 1 hour to digest brain tissue extracellular matrix. Cells were dissociated and titrated to obtain a single cell suspension that was then cultured in suspension as neurospheres within neural basal media supplemented with B27 (Thermo Fisher Scientific, Waltham, MA) and 20 ng/ml basic fibroblast growth factor (FGF-2) and epidermal growth factor (EGF) (Peprotech, Rocky Hill, NJ). Growth media was replaced every two days and neurospheres were passaged every four days or as needed. Cell encapsulation within hydrogels was performed by adding an equal volume of dissociated NSPC suspension in cell media (30,000 cells/μl) to a 10 wt % (M^{O}A)₁₅₅E₆₀ solution in cell media to give a resulting copolymer concentration of 5.0 wt %. This mixture was rapidly combined with an equal volume of 5.0 wt % (M^{O}A)₁₅₅K₆₀ solution in cell media to yield an overall 5.0 wt % cell containing (M^{O}A)₁₅₅E/K₆₀ hydrogel. In a similar manner, a 4.0 wt % K₁₈₀L₂₀ hydrogel control sample in cell media was diluted with an equal volume of cell suspension to yield a final hydrogel concentration of 2.0 wt %. A cell suspension alone in media (15,000 cells/μl) without any hydrogel was also used as a control and baseline. For each of these samples, a 20 μl aliquot was deposited on top of 1.0 wt % agarose gel in media within an Eppendorf tube. The samples were stored in an incubator (37° C., 5% CO₂) and were removed after 1 day for analysis. The samples were diluted 50 fold with PBS, and the cells were pelleted using a microfuge. The Live/Dead® viability/cytotoxicity assay (Thermo Fisher Scientific, Waltham, MA) was employed to quantify the percentages of NSPCs both alive and dead after hydrogel encapsulation. Samples were incubated with Live/Dead stain (2 μM calcein AM and 4 μM EthD-1 in PBS) for 30 min at room temperature. The samples were examined under a Zeiss fluorescence microscope (Carl Zeiss Inc) with filters separating light emission from calcein (live, green, light color) and EthD-1 (dead, red, darker color). Finally, all the live/dead cells were counted using imageJ. The resulting counts were averaged (6 samples of (M^{O}A)₁₅₅E/K₆₀ and 5 samples for both cell control and K₁₈₀L₂₀) and normalized against the cell control.
VI. In certain embodiments, the compositions used in the presently disclosed methods are described in and prepared by the methods disclosed in PCT International Application Publication No. WO 2020/198644, which is hereby incorporated by reference in its entirety.

In some aspects, the composition used in the disclosed methods comprises a first copolypeptide comprising Substructure I, a second copolypeptide comprising Substructure II, a third copolypeptide comprising Substructure III, and water, wherein

Substructure I is depicted as follows:

—Xₘ—Cₚ— or —Cₚ—Xₘ— Substructure I;

Substructure II is depicted as follows:

—Yₙ-A_{q}- or-A_{q}-Yₙ— Substructure II;

Substructure III is depicted as follows:

—Zᵣ-Dₜ- or-Dₜ-Zᵣ— Substructure III;
each instance of X is an amino acid residue independently selected from a non-ionic, hydrophilic amino acid, sarcosine, glycine, and alanine;
each instance of Y is an amino acid residue independently selected from a non-ionic, hydrophilic amino acid, sarcosine, glycine, and alanine;
each instance of Z is an amino acid residue independently selected from a non-ionic, hydrophilic amino acid, sarcosine, glycine, and alanine;
in at least 20% of the instances of C, C is an amino acid residue independently selected from a cationic, hydrophilic amino acid;
in at least 20% of the instances of A, A is an amino acid residue independently selected from an anionic, hydrophilic amino acid;
in at least 20% of the instances of D, D is an amino acid residue independently selected from a non-ionic, hydrophobic amino acid;
m is about 100 to about 600;
n is about 100 to about 600;
r is about 100 to about 600;
p is about 20 to about 200;
q is about 20 to about 200;
t is about 10 to about 200;
at least 90 mol % of the C amino acid residues are (D)-amino acid residues or at least 90 mol % of the C amino acid residues are (L)-amino acid residues;
at least 90 mol % of the A amino acid residues are (D)-amino acid residues or at least 90 mol % of the A amino acid residues are (L)-amino acid residues; and
at least 90 mol % of the D amino acid residues are (D)-amino acid residues or at least 90 mol % of the D amino acid residues are (L)-amino acid residues;
the first copolypeptide and the second copolypeptide are not covalently linked to the third copolypeptide;
the total concentration of the first copolypeptide and the second copolypeptide is about 1% to about 15%; and
the concentration of the third copolypeptide is about 1% to about 10%.

In some aspects, the compositions comprise a first copolypeptide comprising Substructure I′, a second copolypeptide comprising Substructure II′, a third copolypeptide comprising Substructure III′, and water, wherein

Substructure I is depicted as follows:

—Xₘ—Cₚ— or —Cₚ—Xₘ— Substructure I;

Substructure II is depicted as follows:

—Yₙ-A_{q}- or-A_{q}-Yₙ— Substructure II;

Substructure III is depicted as follows:

—Zᵣ-Dₜ- or-Dₜ-Zᵣ— Substructure III;
each instance of X is an amino acid residue independently selected from a non-ionic, hydrophilic amino acid, glycine, and alanine;
each instance of Y is an amino acid residue independently selected from a non-ionic, hydrophilic amino acid, glycine, and alanine;
each instance of Z is an amino acid residue independently selected from a non-ionic, hydrophilic amino acid, glycine, and alanine;
in at least 20% of the instances of C, C is an amino acid residue independently selected from a cationic, hydrophilic amino acid, or a salt thereof;
in at least 20% of the instances of A, A is an amino acid residue independently selected from an anionic, hydrophilic amino acid, or a salt thereof;
in at least 20% of the instances of D, D is an amino acid residue independently selected from a non-ionic, hydrophobic amino acid;
m is about 100 to about 600;
n is about 100 to about 600;
r is about 100 to about 600;
p is about 20 to about 100;
q is about 20 to about 100;
t is about 10 to about 100;
at least 90 mol % of the C amino acid residues are (D)-amino acid residues or at least 90 mol % of the C amino acid residues are (L)-amino acid residues;
at least 90 mol % of the A amino acid residues are (D)-amino acid residues or at least 90 mol % of the A amino acid residues are (L)-amino acid residues; and
at least 90 mol % of the D amino acid residues are (D)-amino acid residues or at least 90 mol % of the D amino acid residues are (L)-amino acid residues;
the first copolypeptide and the second copolypeptide are not covalently linked to the third copolypeptide;
the total concentration of the first copolypeptide and the second copolypeptide is about 1% to about 15%, such as about 1% to about 10%, preferably about 5.0 wt. %; and
the concentration of the third copolypeptide is about 1% to about 10%, such as about 1% to about 5%, preferably about 2.5 wt. %.

In certain embodiments, each instance of X is an amino acid residue independently selected from a non-ionic, hydrophilic amino acid. In certain embodiments, each instance of X is an amino acid residue independently selected from sarcosine, glycine, alanine, methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide. In certain embodiments, each instance of X is an amino acid residue independently selected from methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide. In certain embodiments, at least 90 mol % of the X amino acid residues are (D)-amino acid residues. In certain embodiments, at least 85 mol % of the X amino acid residues are methionine sulfoxide. In certain preferred embodiments, at least 85 mol % of the X amino acid residues are methionine sulfoxide, and the remaining X amino acid residues are alanine. In even further preferred embodiments, about 88 mol % of the X amino acid residues are methionine sulfoxide, and about 12 mol % of the X amino acid residues are alanine.

In certain embodiments, Y is an amino acid residue independently selected from a non-ionic, hydrophilic amino acid. In certain embodiments, each instance of Y is an amino acid residue independently selected from sarcosine, glycine, alanine, methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide. In certain embodiments, each instance of Y is an amino acid residue independently selected from methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide. In certain embodiments, at least 90 mol % of the Y amino acid residues are (D)-amino acid residues. In other embodiments, at least 90% of the Y amino acid residues are (L)-amino acid residues. In certain embodiments, at least 85 mol % of the Y amino acid residues are methionine sulfoxide. In certain preferred embodiments, at least 85 mol % of the Y amino acid residues are methionine sulfoxide, and the remaining Y amino acid residues are alanine. In even further preferred embodiments, about 88 mol % of the Y amino acid residues are methionine sulfoxide, and about 12 mol % of the Y amino acid residues are alanine.

In certain embodiments, each instance of C is an amino acid residue independently selected from a cationic, hydrophilic amino acid, or a salt thereof. In certain embodiments, at least 90% of the C amino acid residues are (D)-amino acid residues. In other embodiments, at least 90% of the C amino acid residues are (L)-amino acid residues. In certain embodiments, each instance of C is lysine, ornithine, or arginine. In certain preferred embodiments, each instance of C is (L)-lysine. In other preferred embodiments, each instance of C is (D)-lysine.

In certain embodiments, each instance of A is an amino acid residue independently selected from an anionic, hydrophilic amino acid, or a salt thereof. In certain embodiments, at least 90% of the A amino acid residues are (D)-amino acid residues. In other embodiments, at least 90% of the A amino acid residues are (L)-amino acid residues. In certain embodiments, each instance of A is glutamic acid or aspartic acid. In certain preferred embodiments, each instance of A is (L)-glutamic acid. In other preferred embodiments, A is (D)-glutamic acid.

In certain embodiments, each instance of Z is an amino acid residue independently selected from a non-ionic, hydrophilic amino acid. In certain embodiments, each instance of Z is an amino acid residue independently selected from sarcosine, glycine, alanine, methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, homomethionine sulfoxide. In certain embodiments, each instance of Z is an amino acid residue independently selected from methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, homomethionine sulfoxide. In certain embodiments, at least 90 mol % of the Z amino acid residues are (D)-amino acid residues. In other embodiments, at least 90 mol % of the Z amino acid residues are (L)-amino acid residues. In certain embodiments, at least 85 mol % of the Z amino acid residues are methionine sulfoxide. In certain embodiments, at least 85 mol % of the Z amino acid residues are methionine sulfoxide, and the remaining Z amino acid residues are alanine. In certain preferred embodiments, at least 85 mol % of the Z amino acid residues are methionine sulfoxide, and the remaining Z amino acid residues are alanine. In certain even further preferred embodiments, about 88 mol % of the Z amino acid residues are methionine sulfoxide, and about 12 mol % of the Z amino acid residues are alanine.

In certain embodiments, each instance of D is an amino acid residue independently selected from a non-ionic, hydrophobic amino acid. In certain embodiments, at least 90% of the D amino acid residues are (D)-amino acid residues. In other embodiments, at least 90% of the D amino acid residues are (L)-amino acid residues. In certain embodiments, each instance of D is leucine, alanine, or phenylalanine. In certain preferred embodiments, each instance of D is (L)-leucine. In other preferred embodiments, each instance of D is (D)-leucine.

In certain embodiments, m is about 100, about 110, about 120, about 130, about 140, about 150, about 160, about 170, about 180, about 190, about 200, about 210, or about 220. In certain preferred embodiments, m is about 120, about 130, about 140, about 150, about 160, about 170, about 180, or about 190.

In certain embodiments, n is about 100, about 110, about 120, about 130, about 140, about 150, about 160, about 170, about 180, about 190, about 200, about 210, or about 220.

In certain preferred embodiments, n is about 120, about 130, about 140, about 150, about 160, about 170, about 180, or about 190.

In certain embodiments, r is about 100, about 110, about 120, about 130, about 140, about 150, about 160, about 170, about 180, about 190, about 200, about 210, or about 220. In certain preferred embodiments, r is about 120, about 130, about 140, about 150, about 160, about 170, about 180, or about 190.

In certain embodiments, p is about 20, about 30, about 40, about 50, about 60, about 70, about 80, about 90, or about 100.

In certain embodiments, q is about 20, about 30, about 40, about 50, about 60, about 70, about 80, about 90, or about 100.

In certain embodiments, t is about 10, about 20, about 30, about 40, about 50, about 60, about 70, about 80, about 90, or about 100.

In certain embodiments, the polydispersity of the first copolypeptide is less than 1.5. In certain embodiments, the polydispersity of the second copolypeptide is less than 1.5.

In certain embodiments, the number of amino acid residues in the first copolypeptide is from about 90% to about 110% of the number of amino acid residues in the second copolypeptide.

In certain embodiments, the composition comprises (M^{O}A)₁₅₅E₃₀, (M^{O}A)₁₅₅E₆₀, (M^{O}A)₁₅₅E₉₀, (M^{O}A)₁₅₅E₁₂₀, (M^{O}A)₁₅₅(rac-E)₆₀, (M^{O}A)₁₅₅K₃₀, (M^{O}A)₁₅₅K₆₀, (M^{O}A)₁₅₅K₉₀, (M^{O}A)₁₅₅K₁₂₀, (M^{O}A)₁₅₀E₅₅, (M^{O}A)₁₅₀K₅₅, or (M^{O}A)₁₅₀L₂₀, or a combination of the foregoing.

In certain embodiments, the composition comprises (M^{O}A)₁₅₀E₅₅, (M^{O}A)₁₅₀K₅₅, or (M^{O}A)₁₅₀L₃₀, or a combination thereof.

In certain embodiments, the composition comprises (M^{O}A)₁₅₀K₅₅ and (M^{O}A)₁₅₀L₂₀.

In certain embodiments, the composition comprises (M^{O}A)₁₅₀E₅₅ and (M^{O}A)₁₅₀L₃₀.

In certain embodiments, the composition comprises (M^{O}A)₁₅₀E₅₅, (M^{O}A)₁₅₀K₅₅, and (M^{O}A)₁₅₀L₃₀.

In certain embodiments, the concentration of the third copolypeptide is about 1% to about 5%. In certain embodiments, the concentration of the third copolypeptide in the composition is about 2.5 wt. %. In certain embodiments, the total concentration of the first copolypeptide and the second copolypeptide is about 1% to about 10%. In certain embodiments, the total concentration of the first copolypeptide and the second copolypeptide in the composition is about 5.0 wt. %. In certain embodiments, the total concentration of the first copolypeptide and the second copolypeptide in the composition is about 5.0 wt. %, and the concentration of the third copolypeptide in the composition is about 2.5 wt. %. In certain embodiments, the total concentration of the first copolypeptide and the second copolypeptide in the composition is about 5.0 wt. %.

In certain embodiments, the molar ratio of C to A is from about 0.95 to about 1.05. In certain embodiments, the molar ratio of X to Y is from about 0.95 to about 1.05. In certain embodiments, the molar ratio of D to A is from about 0.4 to about 0.6.

In certain embodiments, the composition further comprises a salt. In certain embodiments, the concentration of the salt in the composition is less than about 500 mM. In certain embodiments, the concentration of the salt in the composition is from about 100 mM to about 300 mM. In certain preferred embodiments, the salt is NaCl.

In certain embodiments, the composition further comprises a buffer.

In certain embodiments, the composition further comprises a plurality of cells.
VII. In certain embodiments, the compositions used in the presently disclosed methods are disclosed and prepared by the methods disclosed in U.S. Patent Application Publication No. US2021/0330795A1, which is hereby incorporated by reference in its entirety.

In some aspects, the composition used in the disclosed methods comprises a first copolypeptide comprising Substructure I, and a second copolypeptide comprising Substructure II, and water, wherein

Substructure I is depicted as follows:

-A¹ₙ₁-B¹ₘ₁-A¹ₙ₁- Substructure I;

Substructure II is depicted as follows:

—X¹ₙ₂—Y¹ₘ₂—X¹ₙ₂— Substructure II;
each instance of A¹ is an amino acid residue independently selected from a non-ionic hydrophilic amino acid, sarcosine, glycine, and alanine;
in at least 20% of the instances of B¹, B¹ is an amino acid residue independently selected from an anionic hydrophilic amino acid or a salt thereof;
each instance of X¹ is an amino acid residue independently selected from a non-ionic hydrophilic amino acid, sarcosine, glycine, and alanine;
in at least 20% of the instances of Y¹, Y¹ is an amino acid residue independently selected from a cationic hydrophilic amino acid or a salt thereof;
each n1 and n2 is independently about 25 to about 600;
m1 and m2 are independently about 15 to about 600;
at least 75 mol % of the B¹ amino acid residues are (D)-amino acid residues or at least 75 mol % of the B¹ amino acid residues are (L)-amino acid residues;
at least 75 mol % of the Y¹ amino acid residues are (D)-amino acid residues or at least 75 mol % of the Y¹ amino acid residues are (L)-amino acid residues; and
the first copolypeptide and the second copolypeptide are not covalently linked.

In other aspects, the present disclosure provides composition comprising a first copolypeptide comprising Substructure III, and a second copolypeptide comprising Substructure IV, and water, wherein

Substructure III is depicted as follows:

-A¹ₙ₁-B¹ₘ₁-A¹ₙ₃-B¹ₘ₁-A¹ₙ₁- Substructure III;

Substructure IV is depicted as follows:

—X¹ₙ₂—Y¹ₘ₂—X¹ₙ₄—Y¹ₘ₂—X¹ₙ₂— Substructure IV;
each instance of A¹ is an amino acid residue independently selected from a non-ionic, hydrophilic amino acid, sarcosine, glycine, and alanine;
in at least 20% of the instances of B¹, B¹ is an amino acid residue independently selected from an anionic hydrophilic amino acid or a salt thereof;
each instance of X¹ is an amino acid residue independently selected from a non-ionic hydrophilic amino acid, sarcosine, glycine, and alanine;
in at least 20% of the instances of Y¹, Y¹ is an amino acid residue independently selected from a cationic hydrophilic amino acid or a salt thereof;
each n1, n2, n3, and n4 is independently about 25 to about 600;
each m1 and m2 is independently about 15 to about 600;
at least 75 mol % of the B¹ amino acid residues are (D)-amino acid residues or at least 75 mol % of the B¹ amino acid residues are (L)-amino acid residues;
at least 75 mol % of the Y¹ amino acid residues are (D)-amino acid residues or at least 75 mol % of the Y¹ amino acid residues are (L)-amino acid residues; and
the first copolypeptide and the second copolypeptide are not covalently linked.

In certain embodiments, each instance of A¹ is an amino acid residue independently selected from a non-ionic hydrophilic amino acid. In certain embodiments, each instance of A¹ is an amino acid residue independently selected from sarcosine, glycine, alanine, methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide. In certain embodiments, at least 90 mol % of the A¹amino acid residues are (D)-amino acid residues. In other embodiments, at least 90 mol % of the A¹ amino acid residues are (L)-amino acid residues. In certain preferred embodiments, at least 85 mol % of the A¹ amino acid residues are methionine sulfoxide. In certain even further preferred embodiments, at least 85 mol % of the A¹ amino acid residues are methionine sulfoxide, and the remaining A¹ amino acid residues are alanine. In certain most preferred embodiments, about 88 mol % of the A¹ amino acid residues are methionine sulfoxide, and about 12 mol % of the A¹ amino acid residues are alanine.

In certain embodiments, each instance of B¹ is an amino acid residue independently selected from an anionic, hydrophilic amino acid. In certain embodiments, at least 90% of the B¹ amino acid residues are (D)-amino acid residues. In other embodiments, at least 90% of the B¹ amino acid residues are (L)-amino acid residues. In certain preferred embodiments, each instance of B¹ is glutamic acid or aspartic acid. In certain embodiments, each instance of B¹ is (L)-glutamic acid. In other embodiments, each instance of B¹ is (D)-glutamic acid.

In certain embodiments, each instance of X¹ is an amino acid residue independently selected from a non-ionic, hydrophilic amino acid. In certain embodiments, each instance of X¹ is an amino acid residue independently selected from sarcosine, glycine, alanine, methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide. In certain embodiments, at least 90 mol % of the X¹ amino acid residues are (D)-amino acid residues. In other embodiments, at least 90 mol % of the X¹ amino acid residues are (L)-amino acid residues. In certain preferred embodiments, at least 85 mol % of the X¹ amino acid residues are methionine sulfoxide. In certain even further preferred embodiments, at least 85 mol % of the A¹ amino acid residues are methionine sulfoxide, and the remaining X¹ amino acid residues are alanine. In certain most preferred embodiments, about 88 mol % of the X¹ amino acid residues are methionine sulfoxide, and about 12 mol % of the A¹ amino acid residues are alanine.

In certain embodiments, each instance of Y¹ is an amino acid residue independently selected from a cationic, hydrophilic amino acid. In certain embodiments, at least 90% of the Y¹ amino acid residues are (D)-amino acid residues. In other embodiments, at least 90% of the Y¹ amino acid residues are (L)-amino acid residues. In certain preferred embodiments, each instance of Y¹ is lysine, ornithine, or arginine. In certain even further preferred embodiments, each instance of Y¹ is (L)-lysine. In other even further preferred embodiments, each instance of Y¹ is (L)-lysine.

In certain embodiments, each n1 is independently about 20, about 30, about 40, about 50, about 60, about 70, about 80, about 90, or about 100. In certain preferred embodiments, n1 is about 50.

In certain embodiments, each m1 is independently about 10, about 20, about 30, about 40, about 50, or about 60. In certain preferred embodiments, m1 is about 30.

In certain embodiments, each n2 is independently about 20, about 30, about 40, about 50, about 60, about 70, about 80, about 90, or about 100. In certain preferred embodiments, n2 is about 50.

In certain embodiments, each m2 is independently about 10, about 20, about 30, about 40, about 50, or about 60. In certain preferred embodiments, m2 is about 30.

In certain embodiments, n3 is about 50, about 60, about 70, about 80, about 90, about 100, about 110, about 120, about 130, about 140, or about 150. In certain preferred embodiments, n3 is about 100.

In certain embodiments, n4 is about 50, about 60, about 70, about 80, about 90, about 100, about 110, about 120, about 130, about 140, or about 150. In certain embodiments, n4 is about 100.

In certain embodiments, the polydispersity of the first copolypeptide is less than 1.5. In certain embodiments, the polydispersity of the first copolypeptide is greater than 1.0.

In certain embodiments, the polydispersity of the second copolypeptide is less than 1.5. In certain embodiments, the polydispersity of the second copolypeptide is greater than 1.0.

In certain embodiments, the number of amino acid residues in the first copolypeptide is from about 90% to about 110% of the number of amino acid residues in the second copolypeptide.

In certain embodiments, the composition comprises (M^{O}A)₅₀E₃₀(M^{O}A)₅₀, (M^{O}A)₅₀K₃₀(M^{O}A)₅₀, (M^{O}A)₅₀E₃₀(M^{O}A)₁₀₀E₃₀(M^{O}A)₅₀, (M^{O}A)₅₀K₃₀(M^{O}A)₁₀₀K₃₀(M^{O}A)₅₀, (M^{O}A)₄₆E₂₇(M^{O}A)₅₂, (M^{O}A)₄₆K₂₉(M^{O}A)₄₉, (M^{O}A)₄₆E₂₈(M^{O}A)₈₉E₃₁(M^{O}A)₄₈, or (M^{O}A)₄₆K₂₉(M^{O}A)₉₅K₃₁(M^{O}A)₄₆.

In certain embodiments, the total concentration of the first copolypeptide and the second copolypeptide is about 1% to about 15 wt. %. In certain embodiments, the total concentration of the first copolypeptide and the second copolypeptide in the composition is about 5.0 wt. %. In other embodiments, the total concentration of the first copolypeptide and the second copolypeptide in the composition is about 7.0 wt. %. In yet other embodiments, the total concentration of the first copolypeptide and the second copolypeptide in the composition is about 10.0 wt. %.

In certain embodiments, the molar ratio of A¹ to B¹ is about 3:1 or about 4:1.

In certain embodiments, the molar ratio of X¹ to Y¹ is about 3:1 or about 4:1.

In certain embodiments, the composition further comprises a salt. In certain embodiments, the concentration of the salt in the composition is less than about 500 mM. In certain embodiments, the concentration of the salt in the composition is from about 100 mM to about 300 mM. In certain embodiments, the salt is NaCl.

In certain embodiments, the composition further comprises a buffer.

In certain embodiments, the composition further comprises a plurality of cells.

In certain embodiments, the composition has an increased loss modulus (G″) as compared to a composition comprising a diblock polymer comprising the same or substantially similar amino acid residues; wherein both compositions are tested under substantially identical conditions (e.g., the temperature, % wt. of polymer in each composition, and ratio of amino acid components are substantially similar).

In certain embodiments, the composition has an increased storage modulus (G″) as compared to a composition comprising a diblock polymer comprising the same or substantially similar amino acid residues; wherein both compositions are tested under substantially identical conditions (e.g., the temperature, % wt. of polymer in each composition, and ratio of amino acid components are substantially similar).

In certain embodiments, the composition has an increased elasticity as compared to a composition comprising a diblock polymer comprising the same or substantially similar amino acid residues; wherein both compositions are tested under substantially identical conditions (e.g., the temperature, % wt. of polymer in each composition, and ratio of amino acid components are substantially similar).

### Abbreviations

Acetonitrile (MeCN), N-carboxyanhydride (NCA), degree of polymerization (DP), L-methionine (Met), L-methionine residue (M), L-Methionine sulfonium residue (M^{R}), alkyl homocysteine residue (R—C^{H}), glacial acetic acid (AcOH), electrospray ionization-mass spectrometry (ESI-MS), ethanol (EtOH), ethyl acetate (EtOAc), formic acid (HCOOH), diethyl ether (Et₂O), trifluoroacetic acid (TFA), trifluoroacetic anhydride (TFAA), meta-chloroperbenzoic acid (mCPBA), molecular weight cut-off (MWCO), room temperature (RT), equivalents (eq), methanol (MeOH), N,N-dimethylformamide (DMF), broad (br), doublet (d), doublet of doublets (dd), doublet of doublet of doublets (ddd), doublet of multiplets (dm), doublet of quartets (dq), doublet of triplets (dt), pentet (p), quartet (q), septet (sep), sextet (sext) singlet (s), triplet (t), triplet of doublets (td), thin layer chromatography (TLC), acetic anhydride (Ac₂O), Ammonium pyrrolidinedithiocarbamate (APDC), deuterated trifluoroacetic acid (d-TFA), hexafluoroisopropanol (HFiP), pyridine (py), tetrahydrofuran (THF) and triethylamine (TEA).

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease or condition, stabilized (i.e. not worsening) state of disease or condition, preventing spread of disease, delay or slowing progression of disease or condition, amelioration or palliation of the disease state or condition, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other physical condition, is well understood in the art, and includes administration of a composition which reduces the frequency of, or delays the onset of, symptoms of a condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

A “therapeutically effective amount” or a “therapeutically effective dose” of an agent is an amount of an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the polypeptides of the present disclosure can be selected by one of ordinary skilled person in the art to result chemically stable polypeptides which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The definition of each expression, e.g., alkyl, m, n, and the like, when it occurs more than once in any structure, is intended to be independent of its definition elsewhere in the same structure.

Certain compounds contained in compositions of the disclosure may exist in particular geometric or stereoisomeric forms. In addition, polymers of the disclosure may also be optically active. The disclosure contemplates all such compounds, including cis- and trans-isomers, R- and S-enantiomers, diastereomers, (D)-isomers, (L)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included in this disclosure.

If, for instance, a particular enantiomer of compound of the disclosure is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. This disclosure is not intended to be limited in any manner by the permissible substituents of organic compounds.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

For purposes of this disclosure, the chemical elements are identified in accordance with the Periodic Table of the Elements, CAS version, Handbook of Chemistry and Physics, 67th Ed., 1986-87, inside cover.

The term “mixing” refers to any method of contacting one component of a mixture with another component of a mixture, including agitating, blending, combining, contacting, milling, shaking, sonicating, spraying, stirring, and vortexing.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

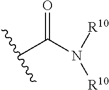

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

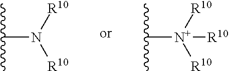

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

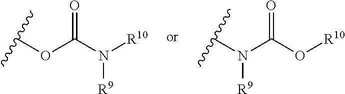

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “polypeptide” refers to a molecule comprising 2 or more amino acids linked by peptide bonds. A polypeptide may be linear or cyclic. A polypeptide may be functionalized or modified at its N-terminus, its C-terminus, or at any of the amino acids within it, including by protecting groups. A polypeptide may contain both natural and unnatural amino acids. “Post-polymerization modification” refers to the action of chemically modifying the amino acids in a polypeptide, the C-terminus, or the N-terminus. A polypeptide may comprise 2 or more amino acids, 5 or more amino acids, 10 or more amino acids, 25 or more amino acids, 50 or more amino acids, or 100 or more amino acids. A polypeptide may be a molecule that is commonly referred to in the art as a “peptide”, an “oligopeptide”, a “polypeptide”, or a “protein”, or any other art-recognized term that satisfies the definition herein. A polypeptide may be part of a larger structure, such as a protein.

The term “silyl” refers to a silicon moiety with three hydrocarbyl moieties attached thereto.

The term “silyloxy” refers to an oxygen moiety with a silyl attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

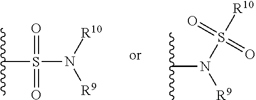

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹¹ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹¹ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

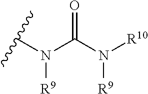

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

The amino acid residue sarcosine (Sar), also called N-methylglycine (MeGly), has the formula:

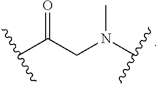 [SMILES: *C(=O)CN(*)C]

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl-protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base polypeptides disclosed herein. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of polypeptides are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of polypeptides for laboratory use, or for subsequent conversion to a pharmaceutically acceptable acid addition salt.

The term “pharmaceutically acceptable basic addition salt” as used herein means any non-toxic organic or inorganic base addition salt of any acid polypeptides represented by Formula I or II. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the polypeptides useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the polypeptides, salts, prodrugs or mixtures thereof (including all possible mixtures of stereoisomers). See, e.g., WO 01/062726.

Furthermore, certain polypeptides which contain alkenyl groups may exist as Z (zusammen) or E (entgegen) isomers. In each instance, the disclosure includes both mixture and separate individual isomers.

Some of the polypeptides may also exist in tautomeric forms. Such forms, although not explicitly indicated in the formulae described herein, are intended to be included within the scope of the present disclosure.

The phrase “pharmaceutically acceptable carrier” as used herein means a pharmaceutically acceptable material, composition or vehicle, such as a liquid or solid filter, diluent, excipient, solvent or encapsulating material useful for formulating an agentfor medicinal or therapeutic or cosmetic use.

The disclosure now being generally described, it will be more readily understood by reference to the following examples which are included merely for purposes of illustration of certain aspects and embodiments of the present disclosure, and are not intended to limit the invention.

### EXAMPLES

The invention is further described in the following examples, which do not limit the scope of the invention described in the claims.

### Example 1: Polyion Complex Hydrogels

### Methods

SEC-RI-MALS measurements were obtained by Malvern Viscotek TDA 305 equipped with UV PDA+RI+RALS+LALS, calibrated with PolyCAL PEO Std Malvern 24.350 Da and using a TSK Gel PWXL G3000-cp cationic column. SEC-RI Column calibration measurements were obtained by Waters Alliance 2695 equipped with W410 RTD+W2487 2 UV k and using TSK Gel PWXL G5000 anionic column. In both SEC instruments, the mobile phase utilized was aqueous NaNO₃ 0.1M with NaN3 0.005% solution.

### Copolymer Synthesis and Properties

The copolymers were obtained via ROP (Ring Opening Polymerization) via the process as generally shown in Scheme 1. Met-NCA and Ala-NCA were dissolved in anhydrous THF under an inert atmosphere. Once the NCAs were completely dissolved, the initiator (isopropyl amine) was added and the mixture was left to stir for three days. After this time, the corresponding 2^{nd} block amino acid-NCA (Lys or Glu) was added, also dissolved in anhydrous THF, and left to stir for a further two days. The copolymer was then precipitated in Et₂O and lyophilized.

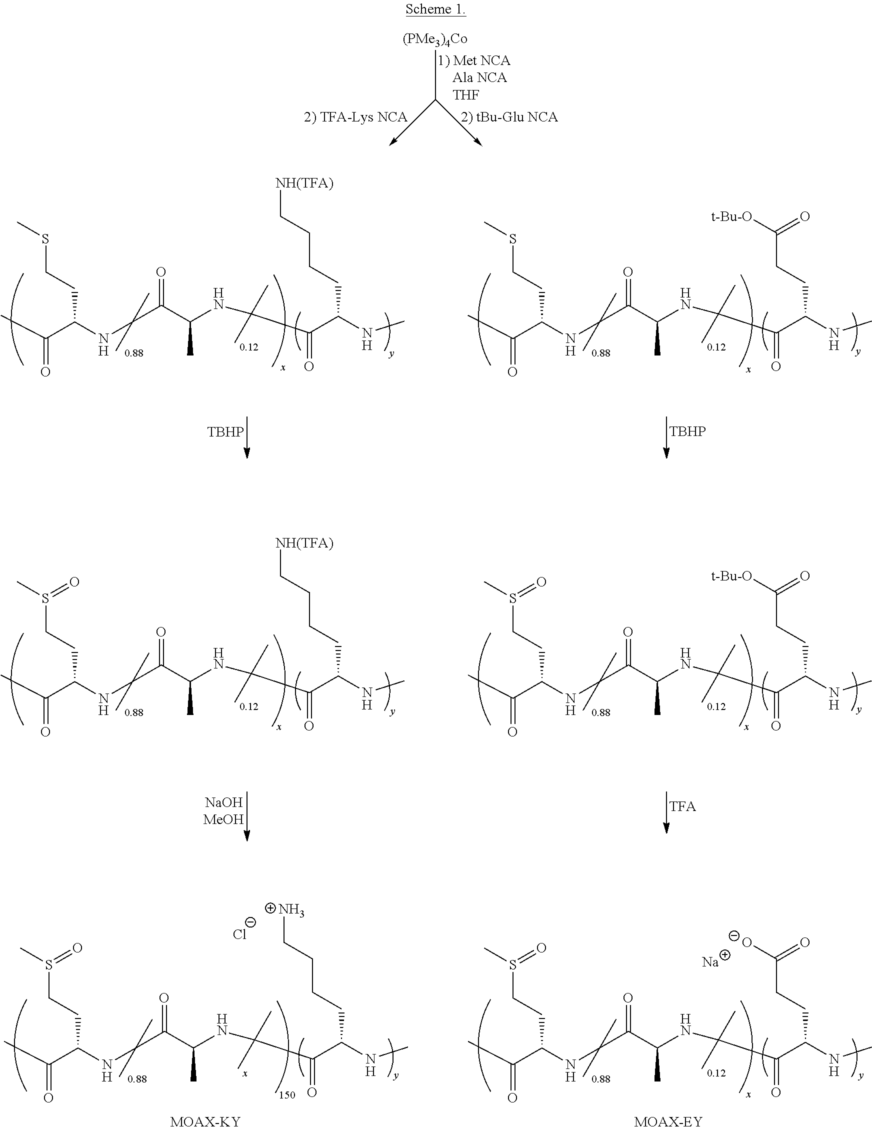

The oxidation reactions were carried out by suspending the copolymer in 16 equivalents of TBHP 80% (tert-butyl hydroperoxide) and 0.2 equivalents of CSA (camphorsulphonic acid) for each Met unit. The oxidation was quenched with Na₂S₂O₃ 0.1M and the product was purified by TFF (Tangential Flow Filtration), and lyophilized.

The (M^{O}A)_{X}-K_{Y} copolymers were then deprotected by dissolving them in TFA and leaving them to stir for three hours. The resulting deprotected copolymer was then precipitated in Et₂O and dried under vacuum. The product was suspended in H₂O and the pH was raised with NaHCO₃ until complete dissolution. The product was purified by TFF, filtered through 0.22 μm and lyophilized.

The (M^{O}A)_{X}-K_{Y} copolymers were deprotected by dissolving in MeOH at 4° C. Aqueous NaOH was added and the reaction was left to stir for 16 hours. After this time, the reaction was acidified with HCl 37% aqueous solution and the product was precipitated in acetone and dried under vacuum. The copolymer was then dissolved in H₂O, purified by TFF, filtered through 0.22 μm and lyophilized.

Target properties and results are shown in Table 1 and Table 2.

TABLE 1
Glutamic acid diblock results

| Product | | | | |
|---|---|---|---|---|
| Batch | (M^{o}A)₁₅₅- | (M^{o}A)₁₅₅- | (M^{o}A)₁₈₀- | (M^{o}A)₁₅₅- |
| Chemical name | E₇₅ | E₈₅ | E₇₅ | E₆₅ |
| Target properties | | | | |
| Degree of Polymerization for | 136 | 136 | 158 | 136 |
| Met(O) | | | | |
| Degree of Polymerization for Ala | 19 | 19 | 22 | 19 |
| Degree of Polymerization for Glu | 75 | 85 | 75 | 65 |
| Molecular weight (Da) | 31104 | 32394 | 34556 | 29814 |
| Analytical Quality | | | | |
| Identity by NMR | Conforms | Conforms | Conforms | Conforms |
| Organic Purity (molar %) by | >99% | >99% | >98% | >98% |
| NMR | | | | |
| Degree of Polymerization for | 154 | 139 | 167 | 143 |
| Met(O) by NMR | | | | |
| Degree of Polymerization for Ala | 25 | 20 | 25 | 21 |
| by NMR | | | | |
| Degree of Polymerization for Glu | 84 | 98 | 80 | 82 |
| by NMR | | | | |
| Molecular Weight (Da) by SEC- | 37782 Da | 39235 Da | 34770 Da | 28821 Da |
| RI* | | | | |
| Sodium by IC (% w/w) | 2.9% | 3.5% | 3.2% | 3.0% |
| Impurities | | | | |
| TFA by IC (% w/w) | <0.01% | 0.01% | <0.01% | <0.01% |

*Values obtained by column calibration

TABLE 2
Lysine diblock results

| Product | | | | |
|---|---|---|---|---|
| Batch | (M^{o}A)₁₅₅- | (M^{o}A)₁₅₅- | (M^{o}A)₁₈₀- | (M^{o}A)₁₅₅- |
| Chemical name | K₆₅ | K₈₅ | K₇₅ | K₇₅ |
| Target properties | | | | |
| Degree of Polymerization for | 136 | 136 | 158 | 136 |
| Met(O) | | | | |
| Degree of Polymerization for Ala | 19 | 19 | 22 | 19 |
| Degree of Polymerization for Lys | 65 | 85 | 75 | 75 |
| Molecular weight (Da) | 29827 | 32411 | 34571 | 31119 |
| Analytical Quality | | | | |
| Identity by NMR | Conforms | Conforms | Conforms | Conforms |
| Organic Purity (molar %) by NMR | >99% | >98% | >99% | >99% |
| Degree of Polymerization for | 159 | 152 | 176 | 141 |
| Met(O) by NMR | | | | |
| Degree of Polymerization for Ala by | 21 | 36 | 30 | 20 |
| NMR | | | | |
| Degree of Polymerization for Lys by | 76 | 103 | 87 | 97 |
| NMR | | | | |
| Molecular Weight (Da) by SEC-RI- | 23453 Da | 23555 Da | 25097 Da | 24141 Da |
| MALS | | | | |
| Polydispersity Index (Mw/Mn) by | 1.01 | 1.01 | 1.03 | 1.03 |
| SEC-RI-MALS | | | | |
| Chloride by IC (% w/w) | 5.4% | 6.7% | 5.3% | 7.7% |
| Impurities | | | | |
| TFA by IC (% w/w) | 0.75% | 1.90% | 0.72% | 0.03% |
| Sodium by IC (% w/w) | 0.09% | 0.06% | 0.07% | 0.10% |

### Rheological Properties

Each PIC hydrogel was generated by mixing equal volumes of copolymer solutions at 7% or 5% w/w (e.g. (M^{O}A)₁₈₀-E₇₅ at 7% mixed with (M^{O}A)₁₈₀-K₇₅ at 7%) in NaCl 0.9%. The mixture was stirred in the vortex for a few seconds and gel formation was confirmed by a 5 second inversion test.

These PIC hydrogels were characterized with the following experiments: Stress sweep; Frequency sweep; Cohesivity test; Osmotic pressure measurement; and pH.

Commercial dermal fillers (Juvederm) were also characterized using the same methods. The rheological characterization experiments have been carried out on a TA discovery HR-1 rheometer with a 40 mm plate-plate geometry, the gap geometry has been set at 500 μm and the sample volume analyzed is 600 μL. Table 3 shows the properties of the different PIC gels and two commercial dermal filers as reference. In the figures it is observed that the elastic modulus (G′), and the loss modulus (G″) of the PIC gels can bracket the values of commercial fillers.

Cohesivity experiments indicate the force necessary to separate the surfaces of 40 mm plates of the rheometer where the gel has been placed between both plates. As can be seen in Table 3, the PIC gels can bracket the values of commercial fillers.

TABLE 3
PIC characterization results.

| | | | G′ at | | G′ = G″ at 1 Hz (Pa) | | Osmotic | |
|---|---|---|---|---|---|---|---|---|
| Copolymer | | | 1 Hz | G″ at | (Viscoelastic | Cohesivity* | Pressure | |
| for PIC | Solvent | w/w | (Pa) | 1 Hz (Pa) | character loss) | (N) | (mOsm/kg) | pH |
| (M^{O}A)₁₅₅- | NaCl | 7% | 45.9 | 19.2 | 45.6 | −0.8 | 404 | 6-7 |
| E/K₆₅ | 0.9% | | | | | | | |
| (M^{O}A)₁₅₅- | NaCl | 7% | 208.5 | 27.9 | 161.2 | −2.2 | 407 | 6-7 |
| E/K₇₅ | 0.9% | | | | | | | |
| (M^{O}A)₁₅₅- | NaCl | 7% | 211.6 | 19.0 | 67.5 | −1.2 | 404 | 6-7 |
| E/K₈₅ | 0.9% | | | | | | | |
| (M^{O}A)₁₈₀- | NaCl | 7% | 919.2 | 55.6 | 374.0 | −10.6 | 406 | 6-7 |
| E/K₇₅ | 0.9% | 5% | 198.2 | 22.0 | 53.0 | −1.9 | 377 | 6-7 |
| Juvederm | PBS 1x | 1.5% | 164.0 | 19.0 | — | −2.0 | 307 | — |
| Volbella | | | | | | | | |
| Juvederm | PBS 1x | 2% | 235.0 | 18.0 | — | −3.6 | 309 | — |
| Voluma | | | | | | | | |

Stronger viscoelastic properties are observed as the polymer lengths increase. This trend can be observed with ionic block (K or E) lengths, but the greatest variation is with the methionine sulfoxide-alanine block (M^{O}A) segment lengths. (M^{O}A)₁₈₀-E/K₇₅PIC hydrogel has the strongest viscoelastic properties of the samples shown.

The pH and osmolarity (osmotic pressure) have also been characterized by potentiometric measurements for pH and by using a freezing point osmometer. Osmolarity and pH measurements can be found in Table 3. pH of PIC gels was found to be between 6 and 7. Osmotic pressure values obtained are higher than commercial dermal fillers due to the presence of added sodium chloride in the formulations, the concentration of which can be lowered as needed.

### Syringe Loading and Extrusion Force

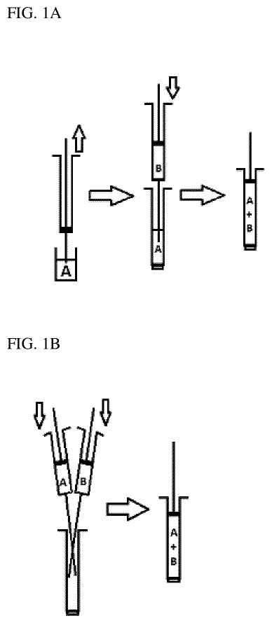

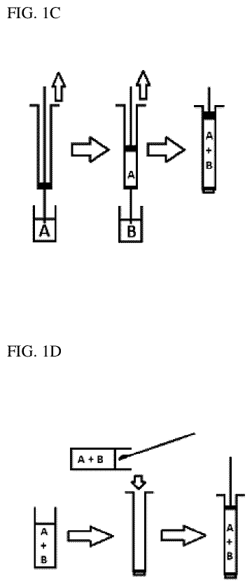

Four different approaches were studied to load the PIC hydrogels into syringes for subsequent injection (FIGS. 1A-1D). Extrusion force tests were carried out to explore which loading method generates a more homogeneous PIC hydrogel. The results in Table 4 show that manual loading gives superior results.

TABLE 4
Extrusion force test for different loading methods

| | | | Extrusion Force (0.3 mm/s) |
|---|---|---|---|
| Copolymer for PIC | w/w | Mixture Method | with 27G needle (N) |
| (M^{o}A)₁₈₀-E/K₇₅ | 7% | Successive Addition | 38-28 |
| | | Inner Addition | 25-12 |
| | | Simultaneous Addition | 33-18 |
| | | Manual loading | 34 |

Once the loading method was defined, new extrusion force tests with 30 G needles were carried out with all the PIC hydrogels and commercial dermal fillers (Table 5). The extrusion force experiments have been carried out on the same rheometer as the previous measurements, in this case, it is an axial force experiment where the rheometer head presses the syringe plunger at 1 mm/s simulating the surgeon's thumb that applies the product. For this experiment, in the case of the PIC gels, the same syringes and needles as the reference fillers (Juvederm) were used.

Despite obtaining higher values than the commercial dermal fillers, all (M^{O}A)₁₅₅ PIC hydrogels gave a constant extrusion force value using 30 G needles. The equipment employed has its force limit above 50 N. Once this pressure is reached, the measurement stops. (M^{O}A)₁₈₀-E/K₇₅ with a 30 G needle reached the force limit but with a 27 G needle gave a constant value below maximum measurement.

TABLE 5
Extrusion force tests for different PICs and commercial dermal fillers.

| Copolymer for PIC | Needle gauge (G) | w/w | Extrusion Force (0.3 mm/s) (N) |
|---|---|---|---|
| (M^{o}A)₁₅₅-E/K₆₅ | 30 | 7% | 40-38 |
| (M^{o}A)₁₅₅-E/K₇₅ | 30 | 7% | 46 |
| (M^{o}A)₁₅₅-E/K₈₅ | 30 | 7% | 47-46 |
| (M^{o}A)₁₈₀-E/K₇₅ | 27 | 7% | 34 |
| | 30 | | >50 |
| | 30 | 5% | >50 |
| Juvederm Volbella | 30 | 1.50% | 18 |
| Juvederm Voluma | 27 | 2% | 20-25 |

### Lidocaine Addition

The compatibility with lidocaine has been tested since it is available in commercial filler formulations at 0.3% w/w. A HPLC method has been developed for the determination of lidocaine in these gels, and can also verify the stability of lidocaine. The analysis was carried out on a C18 reversed phase column with an isocratic H₂O/CH₃OH 90:10 elution with UV-Vis detection. Lidocaine·HCl was utilized for its good solubility in water, and also since this is used in commercial filler formulations. Lidocaine-loaded PIC hydrogels were formulated using the gel preparation method described above but with the M^{O}A-E solution containing 0.6% w/w lidocaine·HCl. When the gel was prepared, the final lidocaine content in the PIC gel was 0.3% w/w.

Comparative results with PICs without lidocaine are shown in Table 6 and Table 7. In general terms, the results obtained were similar for PIC hydrogels with or without lidocaine.

TABLE 6
Osmotic pressure comparison between
unloaded and loaded lidocaine PIC

| | Loaded | Osmotic Pressure |
|---|---|---|
| Copolymer for PIC | Compound | (mOsm/kg) |
| (M^{o}A)₁₅₅-E/K₈₅ | — | 400 |
| (M^{o}A)₁₅₅-E/K₈₅ | Lidocaine HCl | 419 |

TABLE 7
Rheological properties comparison between unloaded and loaded lidocaine PIC

| | | | | G′ = G″ at 1 Hz (Pa) | |
|---|---|---|---|---|---|
| Copolymer for | Loaded | G′ at 1 Hz | G″ at 1 Hz | (Viscoelastic | Cohesivity |
| PIC | Compound | (Pa) | (Pa) | character loss) | (N) |
| (M^{o}A)₁₅₅-E/K₆₅ | Lidocaine | 65.8 | 23.3 | 71.9 | −1.3 |
| | HCl | | | | |
| | 0.3% | | | | |
| | None | 45.9 | 19.2 | 45.6 | −0.8 |
| (M^{o}A)₁₅₅-E/K₇₅ | Lidocaine | 269.6 | 29.3 | 170.2 | −5.3 |
| | HCl | | | | |
| | 0.3% | | | | |
| | None | 208.5 | 27.9 | 161.2 | −2.2 |
| (M^{o}A)₁₅₅-E/K₈₅ | Lidocaine | 531.7 | 54.9 | 95.6 | −2.23 |
| | HCl | | | | |
| | 0.3% | | | | |
| | None | 211.6 | 19.0 | 67.5 | −1.2 |
| (M^{o}A)₁₈₀-E/K₇₅ | Lidocaine | 726.3 | 88.2 | 287.6 | −7.0 |
| | HCl | | | | |
| | 0.3% | | | | |
| | None | 919.2 | 55.6 | 374.0 | −10.6 |

### Example 2: Animal Studies

### Animal Study #1: Preliminary Biocompatibility Rat Subcutaneous Model

### Protocol:

To assess the biocompatibility of the claimed hydrogel fillers in a preclinical in vivo model, subcutaneous injection in rats was performed as previously described (Hillel, Alexander T., et al. “Validation of a small animal model for soft tissue filler characterization.” Dermatologic surgery 38.3 (2012): 471-478., n.d.). Fifteen Male Sprague-Dawley rats (250-300 gm) were injected into the dorsal subcutaneous pocket with the following representative compound: 0.5 mL (M^{O}A)₁₅₅-E/K₆₅ at 9 wt % in 0.9% NaCl

The rats were then scheduled for necropsy with histology according to the following schedule:

TABLE 8

| Group # | Rat # | Termination Day |
|---|---|---|
| 1 | Rats 1-5 | Day 7 |
| 2 | Rats 6-10 | Day 30 |
| 3 | Rats 11-15 | Day 62 |

### Results (Clinical Examination):

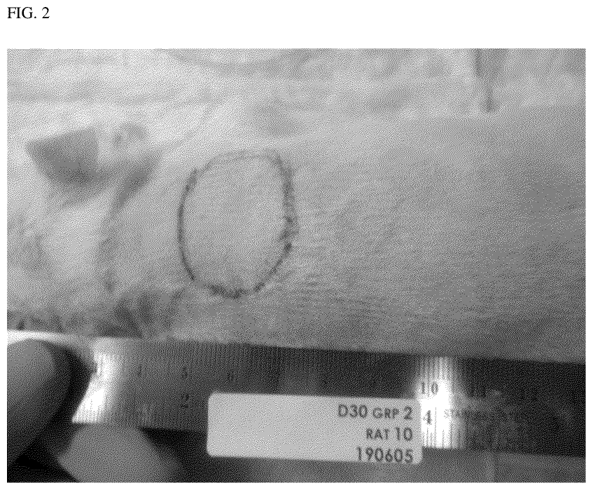

Animals were assessed at days 7, 30, and 62 for clinical irritation or erythema according to the following scale: 1—No erythema (normal); 2—Mild erythema; 3—Moderate erythema; and 4—Severe erythema. FIG. 2 provides an example photograph from an animal in group 2 at day 30 with no observable erythema or irritation at the site of injection (marked with the dark circle).

TABLE 9
Median Clinical (Visual) Assessment
Values of Erythema by Study Day

| | Day 7 | Day 30 | Day 62 |
|---|---|---|---|
| Dermal Filler (n = | 1 | 1 | 1 |
| 5 per timepoint) | | | |

### Results (Histology):

Dermal filler study, group 2-3 injected with test material 30 and 62 days ago ANIMAL SPECIES: Rattus norwegicus/white rat/animals 6-15 day 30-62 GROSS DESCRIPTION: 10 skin samples measuring approximately 4×4 cm MICROSCOPIC DIAGNOSIS:
Animal 6: Subcutis: Fascial infiltrates, mastocytic (mild), lymphoplasmacytic (minimal) and histiocytic (minimal) and fibrocytic (mild), focally extensive with fibrosis (minimal) and rare hair shafts (drag-in from injection);
Animal 7: Subcutis: Fascial infiltrates, mastocytic (minimal), lymphoplasmacytic (minimal), histiocytic (minimal) and fibrocytic (moderate), focally extensive, with fibrosis (mild), panniculus myocyte loss (mild) and rare injection drag-in material
Animal 8: Subcutis: Fascial infiltrates, mastocytic (mild), lymphoplasmacytic (minimal), histiocytic (minimal) and fibrocytic (moderate), focally extensive, with fibrosis (minimal) panniculus myocyte loss (mild);
Animal 9: Subcutis: Fascial infiltrates, mastocytic (mild), lymphoplasmacytic (minimal), histiocytic (minimal) and fibrocytic (moderate), focally extensive, with fibrosis (mild) and panniculus myocyte loss (mild);
Animal 10: Subcutis: Fascial infiltrates, mastocytic (mild), lymphoplasmacytic (minimal), histiocytic (minimal) and fibrocytic (moderate), focally extensive, with fibrosis (mild) and panniculus myocyte loss (mild);
Animal 11: Fascial infiltrates, mastocytic (mild), lymphoplasmacytic (mild) and histiocytic (minimal) and fibrocytic (mild), focally extensive with fibrosis (mild), panniculus myocyte loss (moderate) and rare hair shafts (drag-in from injection);
Animal 12: Fascial infiltrates, mastocytic (mild), lymphoplasmacytic (minimal) and histiocytic (minimal) and fibrocytic (mild), focally extensive with fibrosis (mild) and panniculus myocyte loss (minimal);
Animal 13: Fascial infiltrates, mastocytic (mild), lymphoplasmacytic (mild) and histiocytic (minimal) and fibrocytic (mild), focally extensive with fibrosis (mild) and panniculus myocyte loss (mild);
Animal 14: Fascial infiltrates, mastocytic (mild), lymphoplasmacytic (minimal) and histiocytic (minimal) and fibrocytic (mild), focally extensive with fibrosis (mild) and panniculus myocyte loss (moderate); and
Animal 15: Fascial infiltrates, mastocytic (mild), lymphoplasmacytic (mild) and histiocytic (minimal) and fibrocytic (mild), focally extensive with fibrosis (mild), panniculus myocyte loss (moderate) and rare hair shafts (drag-in from injection).

### SUMMARY

Animals 6-10: These cases had mild to minimal mastocytosis, minimal lymphoplasmacytic inflammation, minimal histiocytic inflammation and minimal to mild fibrosis in the superficial subcutis with multifocal loss of panniculus muscle. No filler material was identified. There are rare cross sections of hair shafts and refractile foreign material (standard injection drag-in with surrounding granulomatous inflammation). Loss of the panniculus muscle is a common finding with subcutaneous injections and is likely unrelated to the test material.

Animals 11-15: These cases had mild to minimal mastocytosis, minimal to mild lymphoplasmacytic inflammation, minimal histiocytic inflammation and minimal to mild fibrosis in the superficial subcutis with multifocal loss of panniculus muscle. No filler material was identified. There are rare cross sections of hair shafts and refractile foreign material (standard injection drag-in with surrounding granulomatous inflammation). Loss of the panniculus muscle is a common finding with subcutaneous injections and is likely unrelated to the test material.

### Animal Study #2: Effectiveness Rat Subcutaneous Model

### Protocol:

To assess the effectiveness and further assess biocompatibility of the claimed hydrogel fillers in a preclinical in vivo model, subcutaneous injection in rats was performed as previously described (Hillel, Alexander T., et al. “Validation of a small animal model for soft tissue filler characterization.” Dermatologic surgery 38.3 (2012): 471-478., n.d.). Sprague-Dawley rats (150-200 gm) were separated into six groups (with 7 animals per group) and each animal was injected directly in the subcutaneous space with one of the following compounds based on group:
Group A: 0.5 mL Juvederm Voluma (Hyaluronic Acid)
Group B: 0.5 mL M^{O}A1₅₅(E/K)₆₅ at 7 wt % in 0.9% NaCl
Group C: 0.5 mL M^{O}Ar₁₅₅(E/K)₇₅ at 7 wt % in 0.9% NaCl
Group D: 0.5 mL M^{O}A₁₅₅(E/K)₈₅ at 7 wt % in 0.9% NaCl
Group E: 0.5 mL M^{O}A₁₈₀(E/K)₇₅ at 7 wt % in 0.9% NaCl
Group F: 0.5 mL M^{O}A₁₈₀(E/K)₇₅ at 5 wt % in 0.9% NaCl

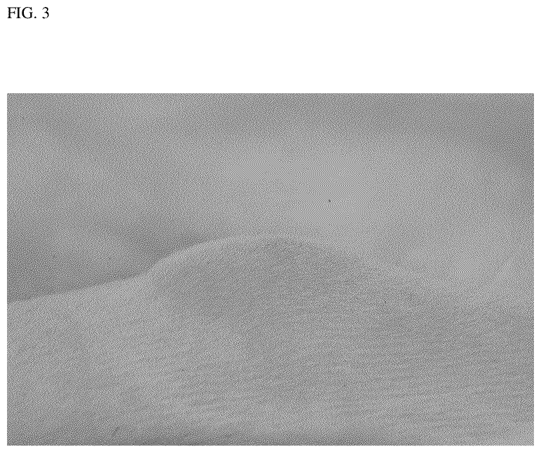

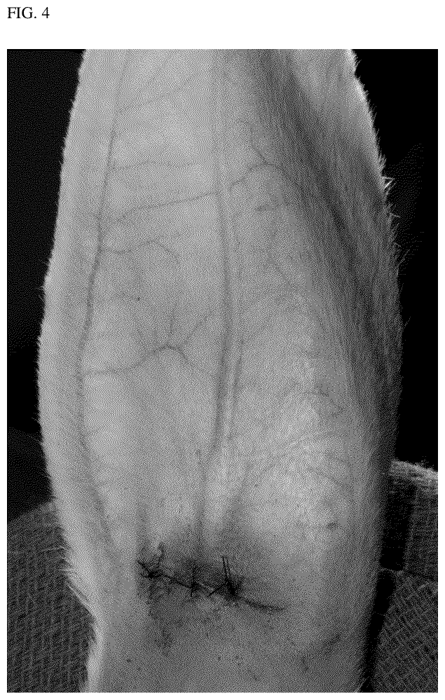

Two rats in each group were sacrificed at day 7 to assess for histologic response to the test material. The remaining five rats in each group were planned to be followed until complete clinical resorption of the test material. Clinical erythema, irritation, and adverse events were similarly observed and recorded. FIG. 3 is an example photo of a palpable lump on the dorsum of an animal in Group A (Hyaluronic Acid Control) on day 7.

### Results (Day 7):

TABLE 10
Gel Persistence

| Animal | Study | | Palpable | Side/Side | Side/Side | Height |
|---|---|---|---|---|---|---|
| Number | Group | Group Description | (Y, N) | (mm) | (mm) | (mm) |
| 1 | A | HA Control | Y | 25 | 25 | 5 |
| 2 | A | HA Control | Y | 25 | 25 | 2.5 |
| 9 | B | MOA₁₅₅(E/K)₆₅ at | N | 25 | 25 | 1 |
| | | 7 wt % in 0.9% NaCl | | | | |
| 10 | B | MOA₁₅₅(E/K)₆₅ at | N | 25 | 25 | 0 |
| | | 7 wt % in 0.9% NaCl | | | | |
| 15 | C | MOA₁₅₅(E/K)₇₅ at | N | 25 | 25 | 0 |
| | | 7 wt % in 0.9% NaCl | | | | |
| 16 | C | MOA₁₅₅(E/K)₇₅ at | N | 25 | 25 | 0 |
| | | 7 wt % in 0.9% NaCl | | | | |
| 23 | D | MOA₁₅₅(E/K)₈₅ at | N | 0 | 0 | 0 |
| | | 7 wt % in 0.9% NaCl | | | | |
| 24 | D | MOA₁₅₅(E/K)₈₅ at | N | 0 | 0 | 0 |
| | | 7 wt % in 0.9% NaCl | | | | |
| 29 | E | MOA₁₈₀(E/K)₇₅ at | N | 0 | 0 | 0 |
| | | 7 wt % in 0.9% NaCl | | | | |
| 30 | E | MOA₁₈₀(E/K)₇₅ at | N | 0 | 0 | 0 |
| | | 7 wt % in 0.9% NaCl | | | | |
| 37 | F | MOA₁₈₀(E/K)₇₅ at | N | 0 | 0 | 0 |
| | | 5 wt % in 0.9% NaCl | | | | |
| 38 | F | MOA₁₈₀(E/K)₇₅ at | N | 0 | 0 | 0 |
| | | 5 wt % in 0.9% NaCl | | | | |

While no photographs were taken, it was noted that the gel deposits in groups B-F persisted for roughly 5 days before complete disappearance of the palpable lump.

### Host Response:

On day 7 there was no detectable erythema, irritation, inflammation, drainage, or infection in any of the study animals in any of the study groups.

### Results (Later Time Points):

Given the lack of remaining palpable lumps in the study groups with PIC gels, the remaining animals in each group were sacrificed at the two-week time point.

### Animal Study #3: Rabbit Arterial Occlusion Study

### Protocol:

A significant advantage of the claimed hydrogel filler is the vascular safety if inadvertently injected intra-arterially. To test the concept that the claimed hydrogel filler does not obstruct arteries, rabbit ears were directly injected intra-arterially with 0.15 cc of material according as previously described in the literature (Nie, Fangfei, et al. “Risk comparison of filler embolism between polymethyl methacrylate (PMMA) and hyaluronic acid (HA).” Aesthetic plastic surgery 43.3 (2019): 853-860.), according to the following groups:

TABLE 11

| | Side | Study | Study Group |
|---|---|---|---|
| Animal # | (L/R) | Group | Description |
| 1 | L | A | HA Control |
| 1 | R | B | MOA₁₅₅(E/K)₆₅ at 7 wt % in 0.9% NaCl |
| 2 | L | C | MOA₁₅₅(E/K)₇₅ at 7 wt % in 0.9% NaCl |
| 2 | R | D | MOA₁₅₅(E/K)₈₅ at 7 wt % in 0.9% NaCl |
| 3 | L | E | MOA₁₈₀(E/K)₇₅ at 7 wt % in 0.9% NaCl |
| 3 | R | A | HA Control |
| 4 | L | B | MOA₁₅₅(E/K)₆₅ at 7 wt % in 0.9% NaCl |
| 4 | R | C | MOA₁₅₅(E/K)₇₅ at 7 wt % in 0.9% NaCl |
| 5 | L | D | MOA₁₅₅(E/K)₈₅ at 7 wt % in 0.9% NaCl |
| 5 | R | E | MOA₁₈₀(E/K)₇₅ at 7 wt % in 0.9% NaCl |
| 6 | L | A | HA Control |
| 6 | R | B | MOA₁₅₅(E/K)₆₅ at 7 wt % in 0.9% NaCl |
| 7 | L | C | MOA₁₅₅(E/K)₇₅ at 7 wt % in 0.9% NaCl |
| 7 | R | D | MOA₁₅₅(E/K)₈₅ at 7 wt % in 0.9% NaCl |
| 8 | L | E | MOA₁₈₀(E/K)₇₅ at 7 wt % in 0.9% NaCl |
| 8 | R | A | HA Control |
| 9 | L | B | MOA₁₅₅(E/K)₆₅ at 7 wt % in 0.9% NaCl |
| 9 | R | C | MOA₁₅₅(E/K)₇₅ at 7 wt % in 0.9% NaCl |
| 10 | L | D | MOA₁₅₅(E/K)₈₅ at 7 wt % in 0.9% NaCl |
| 10 | R | E | MOA₁₈₀(E/K)₇₅ at 7 wt % in 0.9% NaCl |

At day 0, the ears were transilluminated to assess for apparent filler emboli in the vessels. The ears were then clinically assessed at day 7 for ischemic changes.

### Results (Transillumination):

Day 0 transillumination from left ear of rabbit 1 (Hyaluronic acid control) demonstrates impeded blood flow and embolus in the central auricular artery (FIG. 4).

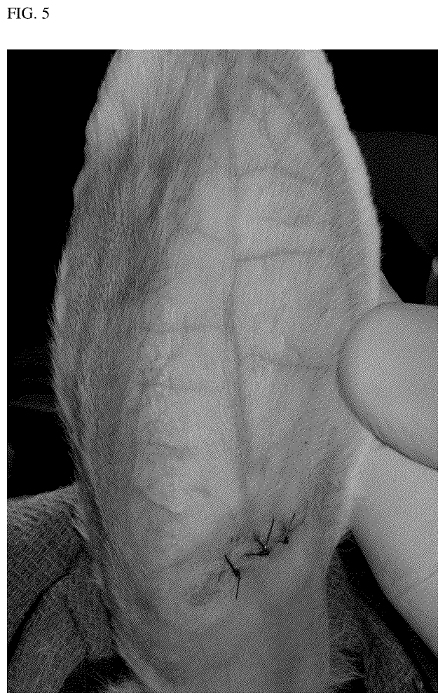

Day 0 transillumination from right ear of rabbit 1 (M^{O}A₁₅₅(E/K)₆₅ at 7 wt % in 0.9% NaCl) demonstrates intact blood flow in the central auricular artery without apparent emboli. These results were representative of the day 0 transillumination experiments. All of the hyaluronic acid ears showed emboli with impeded blood flow, while all of the claimed hydrogel filler injected ears remained patent (FIG. 5).

### Results (Clinical Ischemia):

Animals were then assessed at day 7 for clinically visible ischemic changes.

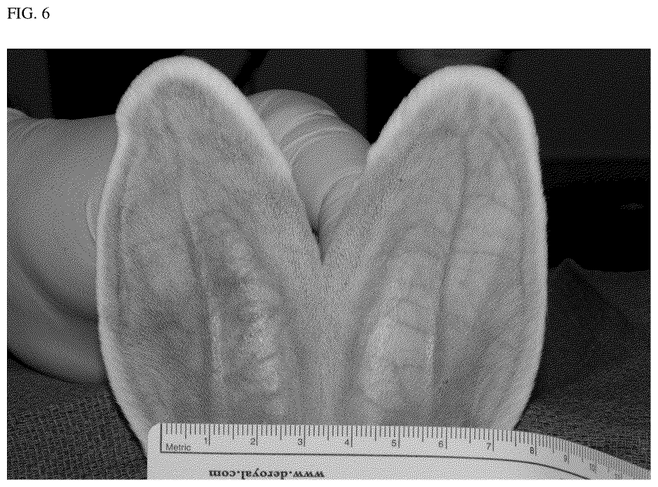

Photo from day 7 from animal 1 depicts ischemic changes in the left ear (hyaluronic acid) compared to right ear (M^{O}A₁₅₅(E/K)₆₅ at 7 wt % in 0.9% NaCl). The ischemic changes are clearly seen as the dusky coloration in the auricular tissue (FIG. 6).

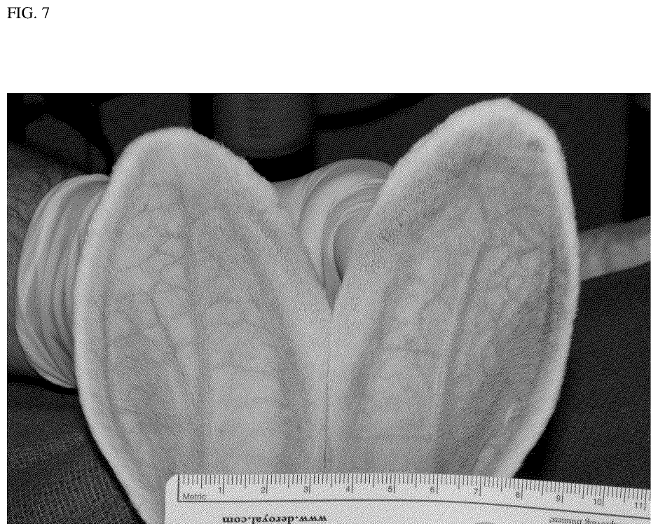

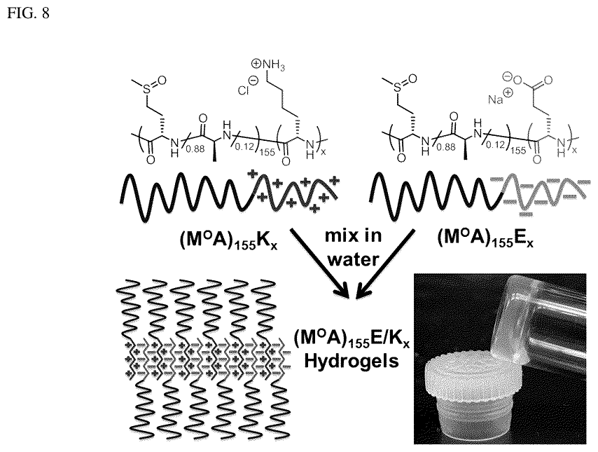

Similarly, photo from day 7 of rabbit 3 demonstrates ischemic changes in the right ear (hyaluronic acid), but no ischemic changes in the left ear (M^{O}A₁₈₀(E/K)₇₅ at 7 wt % in 0.9% NaCl) (FIG. 7).

As with the transillumination studies, these changes were consistent and reproducible in the animals. All of the hyaluronic acid ears demonstrated ischemic changes at day 7, while none of the claimed hydrogel filler injected ears demonstrated ischemic changes.

### Example 3: Polysarcosine Copolymers for PIC

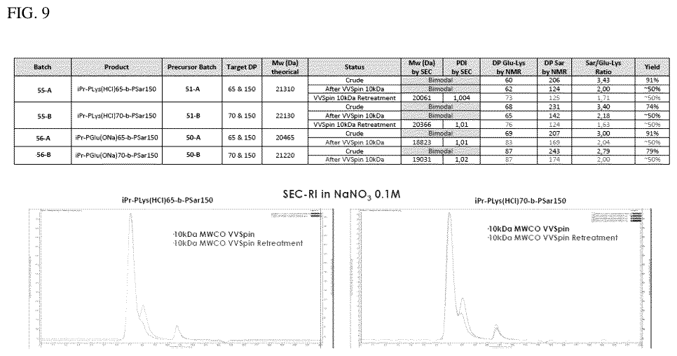

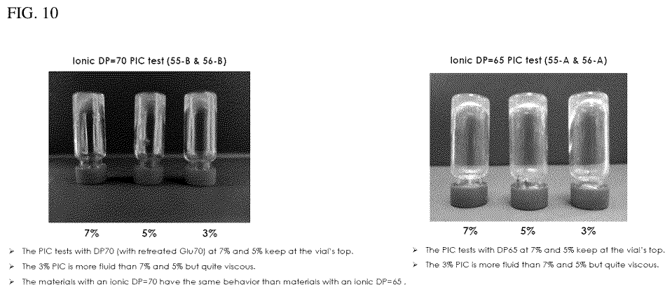

Additional diblock copolypeptide hydrogels (E/Kₓ(Sar)₁₅₀, FIG. 9) were prepared employing sarcosine as the non-ionic amino acid component. Each PIC hydrogel was generated by mixing equal volumes of copolymer solutions at 7%, 5%, or 3% w/w (e.g. iPr-PLys(HCl)₆₅b-PSar₁₅₀ at 7% mixed with iPr-PGlu(ONa)₆₅b-PSar₁₅₀ at 7%) in NaCl 0.9%. The mixture was stirred in the vortex for a few seconds and gel formation was confirmed by a 5 second inversion test. Viscosity of these hydrogels is shown in FIG. 10.

Each PIC hydrogel was generated by mixing equal volumes of copolymer solutions at 7% or 5% w/w (e.g. iPr-PLys(HCl)₆₅b-PSar₁₅₀ at 7% mixed with iPr-PGlu(ONa)₆₅b-PSar₁₅₀ at 7%) in NaCl 0.9%. The mixture was stirred in the vortex for a few seconds and gel formation was confirmed by a 5 second inversion test.

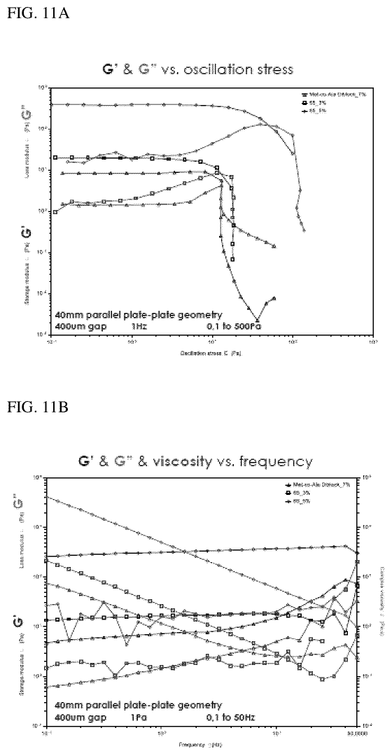

Table 12 and FIGS. 11A-11B shows the properties of different PIC gels, i.e. the elastic modulus (G′) and the loss modulus (G″).

TABLE 12
PIC characterization results.

| | | | | | G′ = G″ 1 Hz |
|---|---|---|---|---|---|
| | | | G′ at 1 | G″ at 1 | (Pa) |
| | | DP ionic | Hz | Hz | (viscoelastic |
| PIC | % wt. | block | (Pa) | (Pa) | character loss) |
| Met-co-Ala Diblock | 9 | 55 | 94.3 | 15.9 | 114.7 |
| (Lys)₅₅-b-(Sar)₁₅₀ + (Glu)₅₅-b- | 5 | 55 | 44.7 | 3.2 | 54.4 |
| (Sar)₁₅₀, both at 5 wt % | | | | | |
| concentration | | | | | |
| (Lys)₇₅-b-(Sar)₁₅₀ + (Glu)₇₅-b- | 5 | 75 | 183.2 | 9.5 | 104.8 |
| (Sar)₁₅₀, both at 5 wt % | | | | | |
| concentration | | | | | |
| (Lys)₅₅-b-(Sar)₁₅₀ + (Glu)₅₅-b- | 7 | 55 | 162.2 | 8.7 | 106.8 |
| (Sar)₁₅₀, both at 7 wt % | | | | | |
| concentration | | | | | |
| (Lys)₇₅-b-(Sar)₁₅₀ + (Glu)₇₅-b- | 7 | 75 | 722.4 | 40.9 | 98.5 |
| (Sar)₁₅₀, both at 7 wt % | | | | | |
| concentration | | | | | |
| Met-co-Ala Diblock (new) | 7 | 55 | 7.2 | 1.5 | 13.2 |
| (Lys)₆₅-b-(Sar)₁₅₀ + (Glu)₆₅-b- | 5 | 65 | 314.3 | 16.2 | 51.2 |
| (Sar)₁₅₀, both at 5 wt % | | | | | |
| concentration | | | | | |
| (Lys)₆₅-b-(Sar)₁₅₀ + (Glu)₆₅-b- | 3 | 65 | 16.5 | 1.6 | 13.1 |
| (Sar)₁₅₀, both at 3 wt % | | | | | |
| concentration | | | | | |

1. A method of treating fine lines or superficial wrinkles in the skin of a subject, comprising administering a composition into a dermal region of the subject which displays the fine lines or superficial wrinkles, thereby treating the fine lines or superficial wrinkles,
wherein the composition comprises a polypeptide hydrogel.

2. The method of claim 1, wherein the dermal region is a tear trough region, a glabellar line, a periorbital region, or a forehead region.

3. A method of treating a skin condition, comprising administering to an individual suffering from the skin condition a composition, wherein the administration of the composition improves the skin condition, thereby treating the skin condition,
wherein the composition comprises a polypeptide hydrogel.

4. The method of claim 3, wherein the skin condition is skin dehydration.

5. The method of claim 4, wherein the composition rehydrates the skin of the subject.

6. The method of claim 3, wherein the skin condition is skin elasticity.

7. The method of claim 6, wherein the composition increases the elasticity of the skin of the subject.

8. The method of claim 3, wherein the skin condition is skin roughness.

9. The method of claim 8, wherein the composition decreases skin roughness in the subject.

10. The method of claim 3, wherein the skin condition is a lack of skin tautness.

11. The method of claim 10, wherein the composition increases skin tautness in the subject.

12. The method of claim 3, wherein the skin condition is a skin stretch line or mark.

13. The method of claim 12, wherein the composition reduces or eliminates the skin stretch line or mark in the subject.

14. The method of claim 3, wherein the skin condition is skin paleness.

15. The method of claim 14, wherein the composition increases skin tone or radiance in the subject.

16. The method of claim 3, wherein the skin condition is skin wrinkles.

17. The method of claim 16, wherein the composition reduces or eliminates skin wrinkles in the subject.

18. A method of preventing skin wrinkles in a subject, comprising administering to the subject a composition, thereby preventing skin wrinkles,
wherein the composition comprises a polypeptide hydrogel.

19. The method of claim 18, wherein the composition makes the skin of the subject resistant to skin wrinkles.

20. The method of any one of claims 1-19, wherein the administration is by subcutaneous injection.

21. The method of any one of claims 1-20, wherein the administration occurs at a depth of less than about 1 mm below the surface of the skin.

22. The method of any one of claims 1-21, wherein the method does not result in arterial occlusion.

23. The method of any one of claims 1-22, wherein the method does not result in unpredictable augmentation.

24. The method of any one of claims 1-23, wherein the method does not result in irritation, for example, chronic irritation.

25. The method of any one of claims 1-24, wherein the composition is soluble in blood.

26. The method of any one of claims 1-25, wherein administration of the composition results in limited swelling.

27. The method of any one of claims 1-26, wherein the administration of the composition results in low immunogenicity.

28. The method of any one of claims 1-27, wherein the composition comprises a first copolypeptide comprising Substructure I, a second copolypeptide comprising Substructure II, and water,
wherein
Substructure I is depicted as follows:
—Xₘ—Cₚ— or —Cₚ—Xₘ— Substructure I;
Substructure II is depicted as follows:
—Yₙ-A_{q}- or-A_{q}-Yₙ— Substructure II;
each instance of X is an amino acid residue independently selected from a non-ionic, hydrophilic amino acid, glycine, alanine, and sarcosine;
each instance of Y is an amino acid residue independently selected from a non-ionic, hydrophilic amino acid, glycine, alanine, and sarcosine;
each instance of C is an amino acid residue independently selected from a cationic, hydrophilic amino acid;
each instance of A is an amino acid residue independently selected from an anionic, hydrophilic amino acid;
m is about 100 to about 600;
n is about 100 to about 600;
p is about 20 to about 200;
q is about 20 to about 200;
at least 90 mol % of the C amino acid residues are (D)-amino acid residues or at least 90 mol % of the C amino acid residues are (L)-amino acid residues; and
at least 90 mol % of the A amino acid residues are (D)-amino acid residues or at least 90 mol % of the A amino acid residues are (L)-amino acid residues.

29. The method of claim 28,
wherein
each instance of X is an amino acid residue independently selected from methionine sulfoxide (M^{o}) and alanine (A);
each instance of Y is an amino acid residue independently selected from methionine sulfoxide (M^{o}) and alanine (A);
each instance of C is the amino acid residue lysine (K); and
each instance of A is the amino acid residue glutamic acid (E).

30. The method of claim 29, wherein about 88 mol % of the X amino acid residues are M^{O}, and about 12 mol % of the X amino acid residues are A; and
about 88 mol % of the Y amino acid residues are M^{O}, and about 12 mol % of the X amino acid residues are A.

31. The method of any one of claims 28-30, wherein m is 155 or 180; p is 55, 65, 75, or 85; n is 155; and q is 55, 65, 75, or 85.33.

32. The method of any one of claims 28-30, wherein m is 155; p is 55, 65, 75, or 85; n is 155; and q is 55, 65, 75, or 85.33.

33. The method of any one of claims 28-32,
wherein
Substructure I is 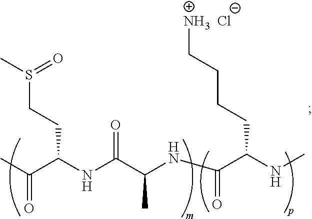
and
Substructure II is

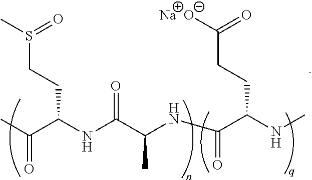

34. The method of any one of claims 28-33, wherein Substructure I is (M^{O}A)₁₈₀-K₇₅; and Substructure II is (M^{O}A)₁₈₀-E₇₅.

35. The method of any one of claims 28-33, wherein Substructure I is (M^{O}A)₁₅₅-K₅₅; and Substructure II is (M^{O}A)₁₅₅-E₅₅.

36. The method of any one of claims 28-33, wherein Substructure I is (M^{O}A)₁₅₅-K₆₅; and Substructure II is (M^{O}A)₁₅₅-E₆₅.

37. The method of any one of claims 28-33, wherein Substructure I is (M^{O}A)₁₅₅-K₇₅; and Substructure II is (M^{O}A)₁₅₅-E₇₅.

38. The method of any one of claims 28-33, wherein Substructure I is (M^{O}A)₁₅₅-K₈₅; and Substructure II is (M^{O}A)₁₅₅-E₈₅.

39. The method of claim 28,
wherein
each instance of X is the amino acid residue sarcosine;
each instance of Y is the amino acid residue sarcosine;
each instance of C is the amino acid residue lysine (K); and
each instance of A is the amino acid residue glutamic acid (E).

40. The method of claim 39, wherein m is 150; p is 65 or 70; n is 150; and q is 65 or 70.
